(19) United States
(12) Patent Application Publication
(10) Pub. No.: US 2025/0000649 A1
(43) Pub. Date: Jan. 2, 2025

(54) DEVICES AND METHODS FOR PROSTHETIC VALVE POSITIONING

(71) Applicant: EDWARDS LIFESCIENCES CORPORATION, Irvine, CA (US)

(72) Inventor: Natanel Simcha Sirote, Zikhron Ya’akov (IL)

(21) Appl. No.: 18/810,753

(22) Filed: Aug. 21, 2024

Related U.S. Application Data
(63) Continuation of application No. PCT/US2023/014101, filed on Feb. 28, 2023.
(60) Provisional application No. 63/315,911, filed on Mar. 2, 2022.

(51) Int. Cl. A61F 2/24

(52) U.S. Cl. CPC A61F 2/2433

(57) ABSTRACT

The present disclosure relates to delivery assemblies that include a measurement device for providing an indication, in real-time, of the position of a prosthetic valve relative to an anatomic structure at the site of implantation. In an example, the delivery assembly comprises a prosthetic valve and a delivery apparatus that includes a handle and a measurement device. The measurement device includes a sensor residing within the handle, a first motion transmitting member coupled to the sensor and extending distally from the handle to a free end thereof, and a second motion transmitting member coupled to the sensor and extending distally from the handle, such that its distal end is releasably coupled to the prosthetic valve. The sensor is configured to sense, in real time, axial movement of the second member's distal end relative to the first member's free end.

### CROSS REFERENCE TO RELATED APPLICATIONS

This application is continuation of International Application No. PCT/US2023/014101, filed Feb. 28, 2023, which claims benefit of U.S. Provisional Application No. 63/315,911, filed on Mar. 2, 2022, the contents of each of which are herein incorporated by reference in their entirety.

### FIELD

The present disclosure relates to delivery assemblies that include a prosthetic valve releasably coupled to a delivery apparatus, and in particular, to delivery assemblies that further include a measurement device for providing an indication, in real-time, of the prosthetic valves position relative to the position of an anatomic structure at the site of implantation.

### BACKGROUND

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures can be performed to repair or replace a heart valve. Surgeries are prone to an abundance of clinical complications, hence alternative less invasive techniques of delivering a prosthetic valve over a catheter and implanting it over the native malfunctioning valve, have been developed over the years.

Different types of prosthetic valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

Accurate placement of a prosthetic valve within the native annulus is important. If the prosthetic valve protrudes too deep into the left ventricle, for example during aortic valve replacement procedures, it may result in hemodynamic disturbances, and may inflict temporary or permanent injury to the conduction system. If the prosthetic valve is placed too high relative to the native annulus, it may dislodge from its location after implantation. Accordingly, a necd exists for improved devices and methods that can provide real-time indication of the prosthetic valve's position, relative to the native annulus, during the implantation procedure.

### SUMMARY

The present disclosure is directed toward delivery assemblies that include a prosthetic valve that can be carried over a delivery apparatus toward a site of implantation, utilizing the delivery apparatus for positioning the prosthetic valve at a desired position within the native annulus, followed by prosthetic valve expansion and delivery apparatus retrieval.

In one representative example, there is provided a delivery assembly comprising a prosthetic valve having an inflow end and an outflow end, and a delivery assembly comprising a handle and a measurement device. The prosthetic valve comprises a frame movable between a radially compressed and a radially expanded configuration. The measurement device comprises a sensor residing within the handle, a first motion transmitting member coupled at a first member proximal end thereof to the sensor and extending from the handle to an opposite first member distal end, and a second motion transmitting member coupled at a second member proximal end thereof to the sensor and extending from the handle to an opposite second member distal end. The first member distal end is a free end. The second member distal end is releasably coupled to the prosthetic valve. The sensor is configured to sense, in real time, axial movement of the second member distal end relative to the first member distal end.

In another representative example, there is provided a delivery assembly comprising a prosthetic valve having an inflow end and an outflow end, and a delivery assembly comprising a handle, a plurality of actuation assemblies, and a measurement device. The prosthetic valve comprises a frame, and a plurality of actuators coupled to support posts of the frame and operable to adjust the frame between a radially compressed and a radially expanded configuration. The actuation assemblies extend distally from the handle and are configured to operate the plurality of actuators. Each actuation assembly comprises an outer sleeve and an actuator driver releasably coupled to a corresponding actuator, wherein the actuator driver extends through the outer sleeve. The measurement device comprises a sensor residing within the handle, a first motion sensor, and one of the plurality of actuator drivers, which is coupled at a driver proximal end thereof to the sensor. The first motion transmitting member is coupled at a first member proximal end thereof to the sensor, and extends from the handle to an opposite first member distal end. The first member distal end is a free end. The sensor is configured to sense, in real time, axial movement of the actuator driver attached thereto relative to the first member distal end.

In another representative example, there is provided a method comprising: positioning a prosthetic valve within a native aortic annulus; and advancing a first motion transmitting member of a delivery apparatus toward an aortic sinus, until a first member distal end of the first motion transmitting member contacts a sinus floor of one of the aortic sinuses. The prosthetic valve has an inflow end and an outflow, and comprises a frame movable between a radially compressed and a radially expanded configuration. The delivery apparatus comprises a handle and a measurement device. The measurement device comprises a sensor residing in the handle and attached to a first member proximal end of the first motion transmitting member, and a second motion transmitting member coupled at a second member proximal end thereof to the sensor, and extending from the handle to an opposite second member distal end. The second member distal end is releasably coupled to the prosthetic valve. The method further comprises sensing, by the sensor, in real time, axial movement of the second member distal end relative to the first member distal end.

In another representative example, there is provided a delivery assembly comprising a prosthetic valve having an inflow end and an outflow end, and a delivery assembly comprising a handle, a plurality of actuation assemblies, and a measurement device. The prosthetic valve comprises a frame, and a plurality of actuators coupled to support posts of the frame and operable to adjust the frame between a radially compressed and a radially expanded configuration. The actuation assemblies extend distally from the handle and are configured to operate the plurality of actuators. Each actuation assembly comprises an outer sleeve and an actuator driver releasably coupled to a corresponding actuator, wherein the actuator driver extends through the outer sleeve. The measurement device comprises a sensor residing within the handle, a first motion sensor, a second motion transmitting member. The first motion transmitting member is coupled at a first member proximal end thereof to the sensor, and extends from the handle to an opposite first member distal end. The second motion transmitting member is coupled at a second member proximal end thereof to the sensor, and extends from the handle to an opposite second member distal end. The first member distal end is a free end. The second member distal end is attached to one of the plurality of actuation assemblies. The sensor is configured to sense, in real time, axial movement of the second member distal end relative to the first member distal end.

In another representative example, there is provided a method comprising: positioning a prosthetic valve within a native aortic annulus; and advancing a first motion transmitting member of a delivery apparatus toward an aortic sinus, until a first member distal end of the first motion transmitting member contacts a sinus floor of one of the aortic sinuses. The prosthetic valve has an inflow end and an outflow, and comprises a frame movable between a radially compressed and a radially expanded configuration. The delivery apparatus comprises a handle, a plurality of actuation assemblies, and a measurement device. The actuation assemblies extend distally from the handle and are configured to operate the plurality of actuators, wherein each actuation assembly comprises an outer sleeve and an actuator driver releasably coupled to a corresponding actuator, the actuator driver extending through the outer sleeve. The measurement device comprises a sensor residing in the handle, a first motion transmitting member coupled at a first member proximal end thereof to the sensor and extending from the handle to an opposite first member distal end, and one of the plurality of actuator drivers, which is coupled at a driver proximal end thereof to the sensor. The method further comprises sensing, by the sensor, in real time, axial movement of the actuator driver attached thereto relative to the first member distal end.

In another representative example, there is provided a method comprising: positioning a prosthetic valve within a native aortic annulus; and advancing a first motion transmitting member of a delivery apparatus toward an aortic sinus, until a first member distal end of the first motion transmitting member contacts a sinus floor of one of the aortic sinuses. The prosthetic valve has an inflow end and an outflow, and comprises a frame movable between a radially compressed and a radially expanded configuration. The delivery apparatus comprises a handle, a plurality of actuation assemblies, and a measurement device. The actuation assemblies extend distally from the handle and are configured to operate the plurality of actuators, wherein each actuation assembly comprises an outer sleeve and an actuator driver releasably coupled to a corresponding actuator, the actuator driver extending through the outer sleeve. The measurement device comprises a sensor residing within the handle, a first motion sensor, a second motion transmitting member. The first motion transmitting member is coupled at a first member proximal end thereof to the sensor, and extends from the handle to an opposite first member distal end. The second motion transmitting member is coupled at a second member proximal end thereof to the sensor, and extends from the handle to an opposite second member distal end. The method further comprises sensing, by the sensor, in real time, axial movement of the second member distal end relative to the first member distal end.

In another representative example, there is provided a delivery assembly comprising a prosthetic valve having an inflow end and an outflow end, and a delivery assembly comprising a handle, a balloon catheter, a push shaft, and a measurement device. The prosthetic valve comprises a frame movable between a radially compressed and a radially expanded configuration. The balloon catheter extends from the handle and comprising an inflatable balloon mounted on a distal end thereof. The push shaft extends from the handle and is disposed over the balloon catheter. The push shaft comprises a push shaft distal end portion opposite to the handle. The measurement device comprises a sensor residing within the handle, a first motion transmitting member coupled at a first member proximal end thereof to the sensor and extending from the handle to an opposite first member distal end, and a second motion transmitting member coupled at a second member proximal end thereof to the sensor and extending from the handle to an opposite second member distal end. The second member distal end is coupled to the push shaft.

The method can further comprise advancing the first motion transmitting member toward an aortic sinus, until the first member distal end contacts a sinus floor of one of the aortic sinuses. The method can further comprise pushing, by the push shaft, the prosthetic valve to position it over the balloon. The method can further comprise positioning the prosthetic valve within the native aortic annulus. The method can further comprise sensing, by the sensor, in real time, axial movement of the second member distal end relative to the first member distal end.

In another representative example, there is provided a method comprising delivering a prosthetic valve in a radially compressed configuration thereof toward a native aortic annulus, while the prosthetic valve is positioned over a balloon catheter of a delivery apparatus, proximal to an inflatable balloon mounted on a distal end of the balloon catheter. The prosthetic valve has an inflow end and an outflow, and comprises a frame movable between the radially compressed and a radially expanded configuration. The delivery apparatus further comprises a handle, a balloon catheter, a push shaft, and a measurement device.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIGS. 1A and 1B are perspective views of one example of a prosthetic valve in a radially expanded configuration with and without a valvular structure, respectively.

FIG. 2 is a detail view of an actuator of the prosthetic valve.

FIGS. 3A and 3B are side views of a proximal end portion and a distal end portion, respectively, of a delivery assembly comprising a delivery apparatus with a prosthetic valve coupled thereto.

FIG. 4 is a cross-sectional view of a shaft assembly of the delivery apparatus, taken along line 4-4 of FIG. 3B.

FIG. 5 is a perspective view of a portion of an actuation assembly of the delivery apparatus.

FIG. 6A is a perspective view of an actuation assembly of the delivery apparatus aligned with an actuator of the prosthetic valve.

FIG. 6B is a perspective view of the actuation assembly engaged with the actuator.

FIG. 6C is a perspective view of the outer sleeve of the actuation assembly engaged with the frame of the prosthetic valve.

FIG. 7 is a perspective view from the inside of a portion of a prosthetic valve with a second motion transmitting member attached to its inflow end.

FIG. 8 is a side view of a proximal end portion of a delivery apparatus that includes a measurement device.

FIG. 9 is a sectional side view of one example of a sensor of the measurement device.

FIGS. 10A and 10B are partial perspective views of a second member distal end adhesively attached and detached, respectively, to and from a portion of the prosthetic valve.

FIGS. 11A and 11B are partial perspective views of a second member distal end threadedly attached and detached, respectively, to and from a portion of the prosthetic valve.

FIGS. 12A-12C are sequential stages of a method of implanting a prosthetic valve.

FIG. 13 is a partial view of another type of a measurement device coupled to a mechanically expandable prosthetic valve.

FIG. 14 is a perspective view on another example of a prosthetic valve in a radially expanded configuration.

FIG. 15 is a side view of another type of a delivery assembly that includes a delivery apparatus and a prosthetic valve.

FIGS. 16A-16C shows sequential stages of a method of advancing a compressed prosthetic valve along a balloon catheter and expanding the prosthetic valve by balloon inflation.

FIG. 17 is a side view of a delivery assembly that includes a measurement device.

FIG. 18 is a sectional side view of an example of a sensor of the measurement device.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

It should be understood that the disclosed examples can be adapted to deliver and implant prosthetic devices in any of the native annuluses of the heart (e.g., the aortic, pulmonary, mitral, and tricuspid annuluses), and can be used with any of various delivery approaches (e.g., retrograde, antegrade, transseptal, transventricular, transatrial, etc.).

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid unduc clutter from having too many reference numbers and lead lines on a particular drawing, some components will be introduced via one or more drawings and not explicitly identified in every subsequent drawing that contains that component.

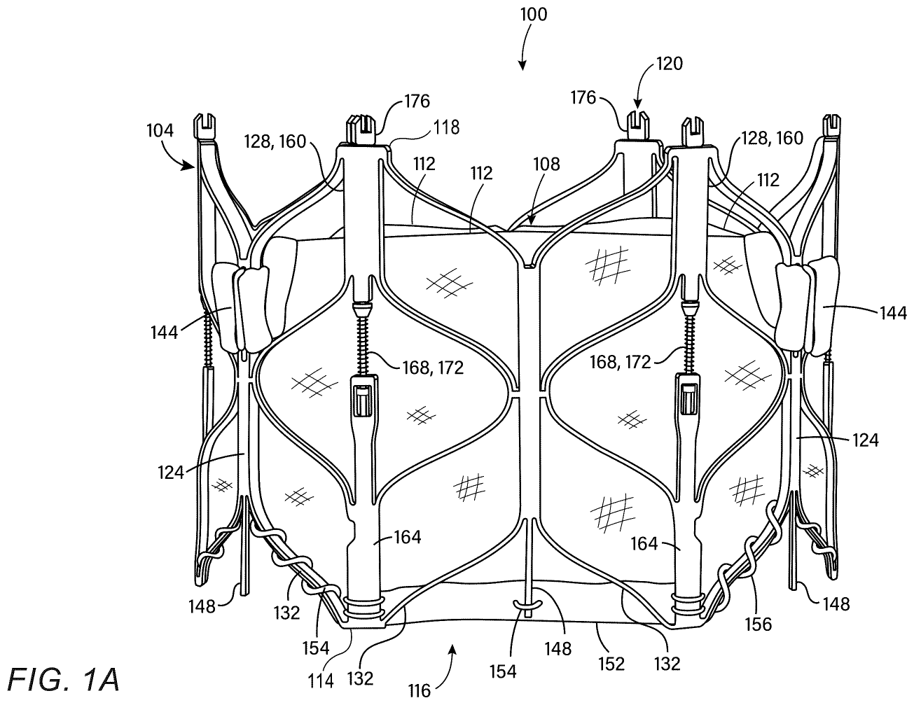

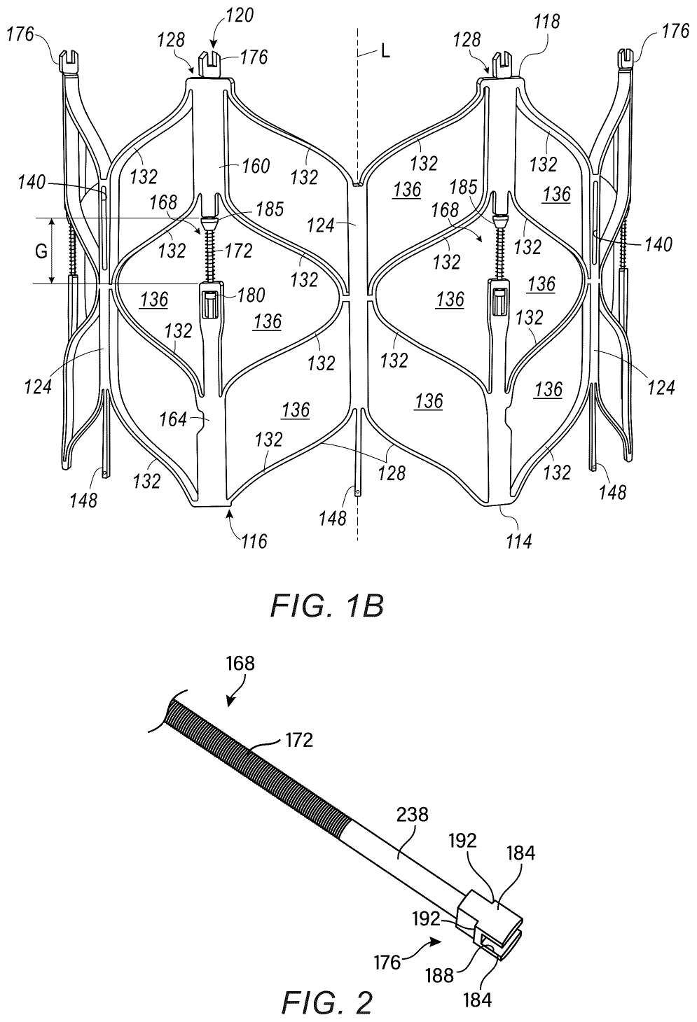

FIG. 1A illustrates a prosthetic valve 100, according to one example. The prosthetic valve 100 can be configured to replace a native heart valve (e.g., aortic, mitral, pulmonary, and/or tricuspid valves). The prosthetic valve 100 is illustrated as a mechanically expandable prosthetic valve that can be radially compressed for delivery to an implantation location within a patient's body and then radially expanded to a working diameter at the implantation location. The prosthetic valve 100 can include a frame 104 having an annular shape. The prosthetic valve 100 can further include a valvular structure 108 supported within and coupled to the frame 104.

In the example, the valvular structure 108 includes one or more leaflets 112 made of flexible material and configured to open and close to regulate blood flow. In one example, the valvular structure 108 can have three leaflets 112, which can be arranged to collapse in a tricuspid arrangement. The leaflets 112 can be made in whole or in part from pericardial tissue (e.g., bovine pericardial tissue), biocompatible synthetic materials, or various other suitable natural or synthetic materials.

As illustrated more clearly in FIG. 1B, the frame 104 has an inflow end 116, an outflow end 120, and a longitudinal axis L extending in a direction from the inflow end 116 to the outflow end 120. The frame 104 can include a plurality of support posts 124, 128 aligned with the longitudinal axis L and spaced along a circumference of the frame 104. In one example, the support posts 124, 128 can be arranged in an alternating manner along the circumference of the frame 104. The frame 104 can further include a plurality of struts 132 extending circumferentially between adjacent support posts 124, 128 and interconnecting the support posts 124, 128. The struts 132 and support posts 124, 128 define cells 136 of the frame 104. As illustrated, the struts 132 can have a curved shape.

As illustrated in FIGS. 1A and 1B, one or more commissure windows 140 can be formed in one or more of the support posts 124. Commissures 144 can be formed at the commissure windows 140 to couple the leaflets 112 to the frame 104. One or more of the support posts 124 can further include cantilevered struts 148 extending to the inflow end 116 of the frame 104. In some examples, the cantilevered struts 148 can extend such that distal ends of the cantilevered struts 148 align with or substantially align with the outflow end 120 of the frame 104.

The prosthetic valve 100 can further include one or more skirts or sealing members. For example, the prosthetic valve 100 can include an inner skirt (not shown in FIGS. 1A-B), mounted on the radially inner surface of the frame 104. The inner skirt can function as a scaling member to prevent or decrease perivalvular leakage, to anchor the leaflets 112 to the frame 104, and/or to protect the leaflets 112 against damage caused by contact with the frame 104 during crimping and during working cycles of the prosthetic valve 100. The prosthetic valve 100 can further include an outer skirt (not shown in FIGS. 1A-B) mounted on the outer surface of the frame 104. The outer skirt can function as a sealing member for the prosthetic valve 100 by scaling against the tissue of the native valve annulus and helping to reduce paravalvular leakage past the prosthetic valve 100. The inner and outer skirts can be formed from any of various suitable biocompatible materials, including any of various synthetic materials, including fabrics (e.g., polyethylene terephthalate fabric) or natural tissue (e.g., pericardial tissue). Further details regarding the use of skirts or scaling members in prosthetic valves can be found, for example, in U.S. Patent Application No. 62/854,702 and PCT Patent Application No. US2020/024559, each of which is incorporated by reference herein.

In some cases, as shown in the example illustrated in FIG. 1A, inflow edge portions 152 of the leaflets 112 can be attached to the cantilevered struts 148 (e.g., by sutures 154) and/or to selected struts 132 of the frame 104 (e.g., using sutures 156). Alternatively or additionally, cantilevered struts 148 can prevent or mitigate portions of an outer skirt from extending radially inwardly and thereby prevent or mitigate any obstruction of flow through the outflow end 120 of the frame 104 caused by the outer skirt. The cantilevered struts 148 can further serve as supports to which portions of the inner and/or outer skirts can be coupled. For example, sutures used to connect the inner and/or outer skirts can be wrapped around the cantilevered struts 148 and/or can extend through apertures formed at end portions of the cantilevered struts 148.

While leaflets 112 are shown in the example illustrated in FIG. 1A to be sutured directly to struts 132 of the frame 104, in other implementation, proximal edge portions of the leaflets can be sutured to an inner skirt generally along the scallop line. The inner skirt can in turn be sutured, via one or more sutures, for example, to adjacent struts 132 of the frame 104.

In one example, the frame 104 can be adjusted between a radially expanded configuration and a radially compressed configuration by deflecting the struts 132. In one example, the frame 104 (e.g., the posts and struts) can be made of biocompatible plastically-expandable materials that will allow the frame 104 to be adjusted between the radially expanded configuration and radially compressed configuration. Suitable examples of plastically-expandable materials that can be used in forming the frame 104 include, but are not limited to, stainless steel, cobalt chromium alloy, and/or nickel titanium alloy (which can also be referred to as “NiTi” or “nitinol”).

Referring to FIG. 1B, in one example, one or more actuators 168 can be coupled to the support posts 128 and used to adjust the frame 104 between the radially expanded configuration and the radially compressed configuration. In one example, each support post 128 can include an upper post member 160 and a lower post member 164 (the terms “upper” and “lower” are relative to the orientation of the frame 104 in FIG. 1A) aligned with the longitudinal axis L of the frame 104 and having opposing ends separated by a gap G. The respective actuator 168 can be coupled to the post members 160, 164 and operable to increase or decrease the gap G in order to radially compress or expand the frame 104. Struts 132 can converge with upper post members 160 to define outflow apices 118 at the outflow end 120. Struts 132 can similarly converge with lower post members 164 to define inflow apices 114 at the inflow end 116.

In one example, the actuator 168 can include an actuator rod 172 with an attached actuator head 176. In the examples illustrated in FIG. 1B, the actuator rod 172 extends through or into the post members 160, 164 and across the gap G. In the example illustrated in FIG. 1B, the actuator rod 172 is inserted into the upper post member 160 from the outflow end 120, and the actuator head 176 is disposed or retained at the outflow apex of the upper post member 160.

In some examples, the actuator rod 172 is externally threaded. As illustrated in FIG. 1B, the lower post member 164 can include a nut 180 with an internal thread to threadedly engage the actuator rod 172. In this case, the actuator rod 172 can be axially translated by rotating the actuator rod 172 relative to the nut 180. In some examples, the actuator rod 172 can be freely slidable relative to the upper post member 160. In other examples, the actuator rod 172 can threadedly engage the upper post member 160. The term “axially translated”, as used herein, refers to translation along an axis coinciding with or parallel to longitudinal axis L.

As illustrated in FIG. 2, the actuator head 176 can include a pair of protrusions 184 forming a slot 188. The actuator head 176 can further include one or more shoulders 192. As will be further described, an actuation assembly of the delivery apparatus can releasably engage the actuator head 176 via the slot 188 and shoulders 192.

Referring to FIG. 1B, in one scenario, the actuator rod 172 can be rotated in a first direction to move the upper post member 160 towards the lower post member 164 and thereby decrease the size of gap G, which can have the effect of radially expanding the frame 104. In another scenario, the lower post member 164 may be held steady while the actuator rod 172 is rotated in a second direction to move the upper post member 160 away from the lower post member 164 and thereby increase the size of gap G, which can have the effect of radially compressing the frame 104.

The actuator rod 172 also can include a stopper 185 (e.g., in the form of a nut, washer or flange) disposed thereon. The stopper 185 can be disposed on actuator rod 172 such that it sits within the gap G. Further, the stopper 185 can be integrally formed on or fixedly coupled to the actuator rod 172 such that it does not move relative to the actuator rod 172. Thus, the stopper 185 can remain in a fixed axial position on the actuator rod 172 such that it moves in lockstep with the actuator rod 172.

When the actuator rod 172 is rotated in a direction configured to collapse the prosthetic valve, the stopper 185 moves toward the outflow end 120 of the frame until the stopper 185 abuts the inflow end of the upper post member 160. Upon further rotation of the actuator rod 172, the stopper 185 can apply a proximally directed force to the upper post member 160 to radially compress the frame 104. Specifically, during crimping/radial compression of the prosthetic valve 100, the actuator rod 172 can be rotated in a direction that causes the stopper 185 to push against (i.e., provide a proximally directed force to) the inflow end of the upper post member 160, thereby causing the upper post member 160 to move away from the lower post member 164, and thereby axially elongating and radially compressing the prosthetic valve 100.

In an alternative implementation, some of the actuator rods 172 can be rotated in one direction while the other actuator rods 172 are rotated in an opposite direction simultaneously to either radially expand the frame or radially compress the frame. This counter-rotation of the actuator rods can be used to help reduce the likelihood of the entire frame 104 rotating about the longitudinal axis L during rotation of the actuator rods 172 about their respective axes (e.g., when radially expanding the frame 104).

Additional examples of mechanically expandable valves can be found in International Application No. PCT/US2021/052745 and U.S. Provisional Applications Nos. 63/209,904 and 63/282,463, which are incorporated by reference herein.

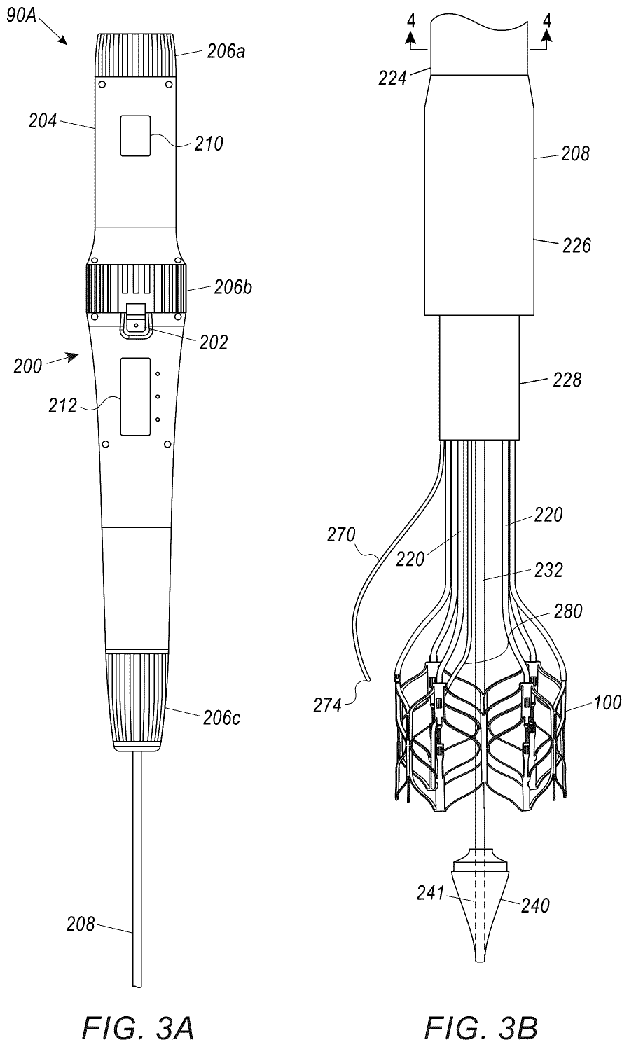

FIGS. 3A and 3B illustrate a delivery assembly 90A, which can includes a prosthetic valve 100 and a delivery apparatus 200, according to one example. The delivery apparatus 200 can be used to deliver the prosthetic valve 100 to an implantation location within a patient's body. The delivery apparatus 200 includes a handle 204 and a shaft assembly 208 coupled to the handle 204. The delivery apparatus 200 can further include one or more actuation assemblies 220 that can be used to releasably couple the prosthetic valve 100 to a distal end portion of the shaft assembly 208 and to radially expand and/or compress the prosthetic valve 100.

In addition, the delivery apparatus 200 can optionally include a sensor data unit 210, and one or more visual or auditory informative elements configured to provide visual or auditory information and/or feedback to a user or operator of delivery apparatus 200, such as a display 212, LED lights, speakers (not shown) and the like.

The prosthetic valve 100 is shown in an expanded configuration in FIG. 3B. To facilitate delivery of the prosthetic valve 100 to an implantation location, the delivery apparatus 200 (and/or other crimping devices) can be used to move the prosthetic valve 100 from a radially expanded, functional configuration to a radially compressed, delivery configuration. Once at the implantation location, actuation drivers of the actuation assemblies 220 can operate the actuators 168 of the prosthetic valve 100 to radially expand the prosthetic valve 100 to a working diameter.

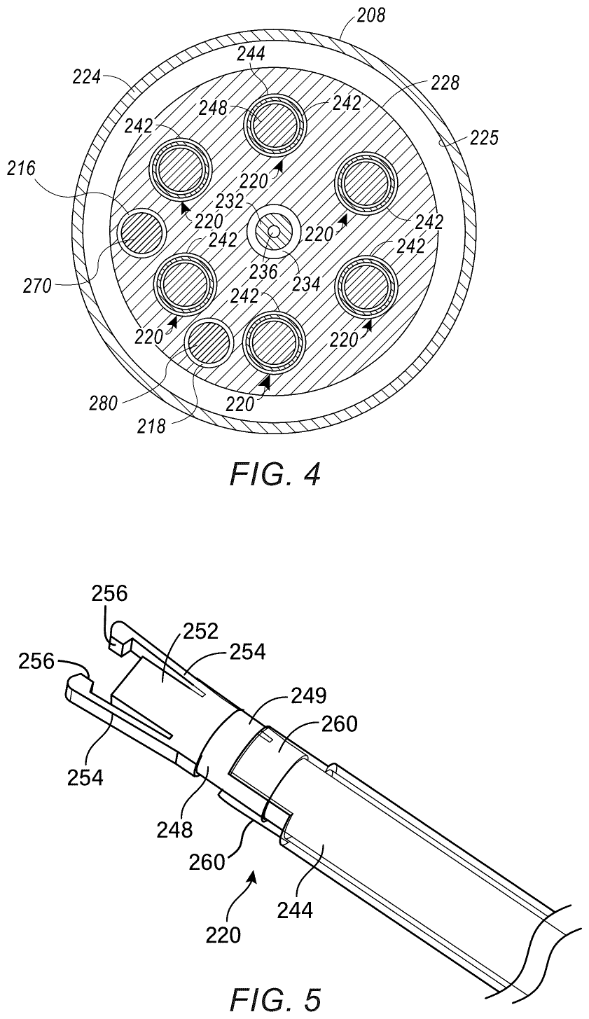

As illustrated in FIGS. 3B and 4, the shaft assembly 208 can include an outer delivery shaft 224 having a lumen 225 extending along the entire length of the shaft. The shaft assembly 208 can include a multi-lumen delivery shaft 228 extending through the lumen 225 and having lumens 234, 242. The shaft assembly 208 can include a nosecone shaft 232 extending through the lumen 234. The actuation assemblies 220 can extend through the lumens 242. The lumen 234 can be centrally disposed within the multi-lumen delivery shaft 228, and the lumens 242 can be angularly spaced apart (uniformly or non-uniformly) about a central axis of the multi-lumen delivery shaft 228 and disposed around the lumen 234.

In one example, the proximal end portion of the nosecone shaft 232 extends into a cavity of the handle 204, and the distal end portion of the nosecone shaft 232 extends distally from the distal end of the multi-lumen delivery shaft 228 (as shown in FIG. 3B). The prosthetic valve 100 can be disposed around the distal end portion of the nosecone shaft 232 when releasably coupled to the actuation assemblies 220.

The nosecone shaft 232 can define a guidewire lumen 236 for receiving a guidewire. As shown in FIG. 3B, a nosecone 240 can be attached to a distal end of the nosecone shaft 232. The nosecone 240 can have a central opening 241 that is aligned and connected to the guidewire lumen 236. During an implantation procedure, a guidewire can be initially inserted into a patient's vasculature. The proximal end of the guidewire can be inserted into the central opening 241 of the nosecone 240 to allow the delivery apparatus 200 to be advanced through the patient's vasculature to an implantation location over the guidewire.

Delivery apparatus 200 can further include a first motion transmitting member 270 extending distally from the handle 204 and terminating at a first member distal end 274, and a second motion transmitting sensor 280 extending distally from the handle 204 and terminating at a second member distal end 284. First member distal end 274 is a free end, not attached to any components of the delivery apparatus 200 or prosthetic valve 100, while second member distal end 284 is releasably attachable to the prosthetic valve 100. Any of the first motion transmitting member 270 and/or second motion transmitting member 280 can extend through the shaft assembly 208, including through any of the outer delivery shaft 224 and/or the multi-lumen delivery shaft 228. The terms “releasably coupled” or “releasably attached”, as used herein, are interchangeable, and refer to two components coupled in such a way that they are coupled together and can be separated without plastically deforming either of the components.

FIGS. 3B and 4 illustrated an example of a multi-lumen delivery shaft 228 that includes additional lumens 216, 218. The first motion transmitting member 270 can extend through lumen 216. The second motion transmitting member 280 can extend through lumen 218. In one example, the first motion transmitting member 270 and the second motion transmitting member 280 extend distally from the distal end of the multi-lumen delivery shaft 228 (as shown in FIG. 3B). In alternative implementation, only one or none of the first motion transmitting member 270 and/or the second motion transmitting member 280 extends through multi-lumen delivery shaft 228. For example, in some implementations, the first motion transmitting member 270 can extend through the lumen 225 of the outer delivery shaft 224, for example between the inner surface of outer delivery shaft 224 and outer surface of multi-lumen delivery shaft 228, while the second motion transmitting member 280 can extend through lumen 218 of multi-lumen delivery shaft 228. In yet other alternative implementations, both the first motion transmitting member 270 and the second motion transmitting member 280 extend through the lumen 225, such as between the inner surface of outer delivery shaft 224 and outer surface of multi-lumen delivery shaft 228 (alternative implementations not shown).

The first motion transmitting member 270 can be concealed within shaft assembly 208 during delivery, and can have its distal end exposed when reaching closer to the site of implantation, for example by extending from one of the shafts of shaft assembly 208, such as multi-lumen delivery shaft 228 or outer delivery shaft 224, while its first member distal end 274 is free-ended and is not attached to the prosthetic valve 100. Moreover, when the first member distal end 274 is axially positioned between the level of the outflow end 120 and the inflow end 116, it is disposed out of the prosthetic valve 100, away from longitudinal axis L, and is free to move radially away from the prosthetic valve 100 (such as away from frame 104). The term “axially positioned” refers to a position between two different regions which are spaced apart from each other along an axis that coincides with or is parallel to the longitudinal axis L.

The first motion transmitting member 270 extends distally from a shaft of shaft assembly 208, such as multi-lumen delivery shaft 228 or outer delivery shaft 224, such that the first member distal end 274 can be releasably coupled to the prosthetic valve 100.

Although FIGS. 3A-4 are described herein together, this is not meant to be limiting in any way, and delivery apparatus 200 can be provided without optional outer delivery shaft 224, delivery capsule 226, multi-lumen delivery shaft 228, nosecone 240 and nosecone shaft 232, sensor data unit 210, and/or display 212, without exceeding the scope of the disclosure.

FIG. 5 illustrates a distal end portion of the actuation assembly 220. Each actuation assembly 220 can include an outer sleeve 244 and an actuator driver 248 extending through the outer sleeve 244. In the example, the actuator driver 248 includes a distal head having a central protrusion 252 and one or more flexible elongated elements 254. The central protrusion 252 can be configured to extend into the slot 188 (shown in FIG. 2) of the actuator head 176 of an actuator 168 of the prosthetic valve. The flexible elongated elements 254 can have radial protrusions 256 configured to engage the shoulders 192 (shown in FIG. 2) of the actuator head 176.

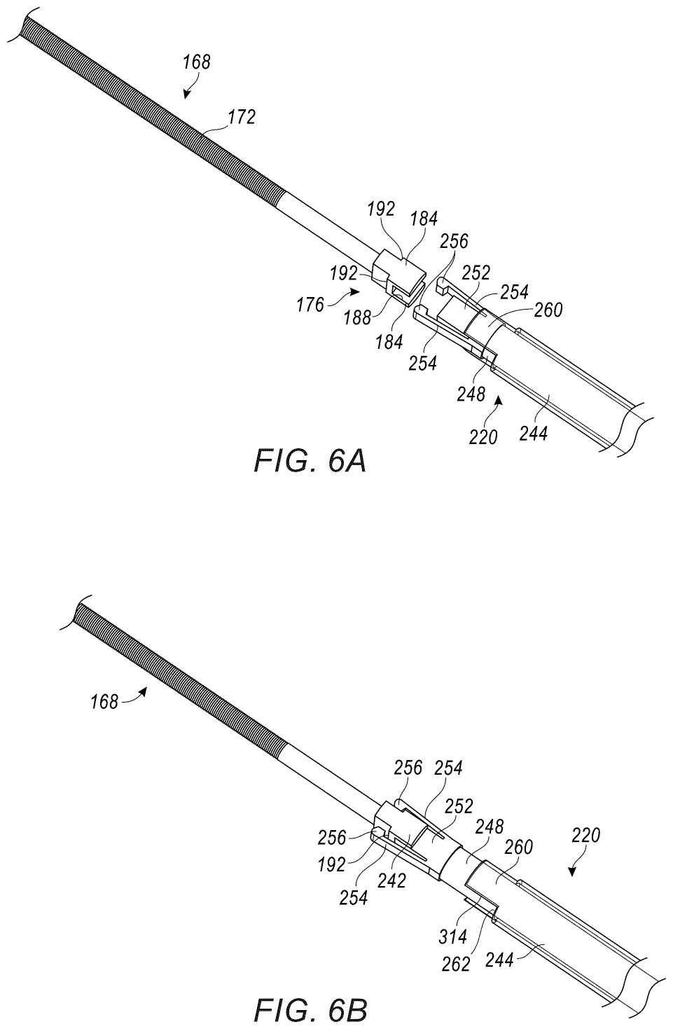

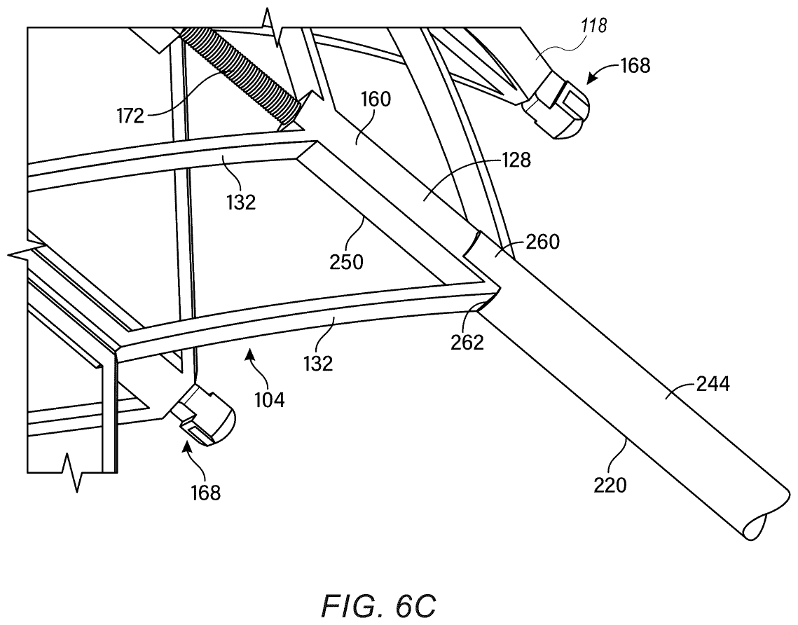

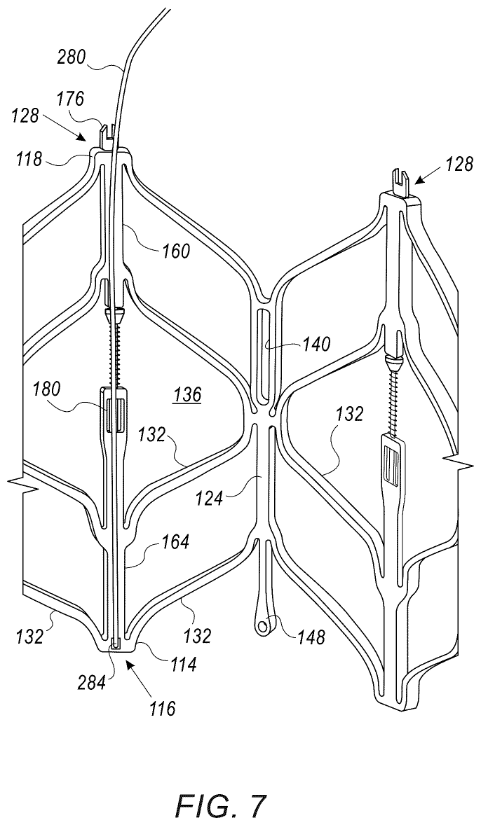

FIGS. 6A-6C illustrate engagement of an actuation assembly 220 with a respective actuator 168. Initially, the distal end portion of the actuation assembly 220 is aligned with the actuator head 176 of the actuator 168, as shown in FIG. 7A. The distal end portion of the actuator driver 248 is then advanced such that the central protrusion 252 of the actuator driver 248 is disposed within the slot 188 of the actuator head 176 of the actuator 168. When the central protrusion 252 is engaged with the slot 188, the flexible elongated elements 254 are disposed at the sides of the actuator head 176, and the radial protrusions 256 of the flexible elongated elements 254 are positioned distally to the shoulders 192 on the actuator head 176, as shown in FIG. 6B.

The outer sleeve 244 can be advanced over the distal end portion of the actuator driver 248 to radially compress the flexible elongated elements 254 against the actuator head 176 until the radial protrusions 256 abut the shoulders 192, thereby coupling the actuator driver 248 to the actuator 168. The outer sleeve 244 can be further advanced until the outer sleeve 244 engages the frame 104, as illustrated in FIG. 6C.

The outer sleeve 244 can have first and second support extensions 260 defining gaps or notches 262 between the extensions 260. As illustrated in FIG. 6C, the support extensions 260 can be oriented such that when the actuation assembly 220 is coupled to a respective actuator 168, the support extensions 260 extend partially over a proximal end portion of the upper post arm 160 of the respective support post 128. The engagement of the support extensions 260 with the frame 104 can counteract rotational forces applied to the frame 104 by the actuator rods 172 during expansion of the frame 104.

Various other coupling mechanisms can be used to releasably couple the prosthetic valve to the actuation assembly of the delivery apparatus. For example, additional coupling mechanisms are described in U.S. Application No. 63/194,285 and PCT Application No. US2021/052745, which are incorporated by reference herein.

As shown in FIG. 3A, the handle 204 can include one or more knobs that can be configured to perform various functions of the delivery apparatus 200 to deliver the prosthetic valve 100 to an implantation location within a patient's body. In one example, the handle 204 can include a first knob 206a, a second knob 206b, and a third knob 206c. In one example, the knobs 206a, 206b, 206c can be knobs that are rotatable about a central longitudinal axis of the handle 204. The handle 204 can include other knobs that can be rotatable or slidable, such as a safety knob 202.

In an example, the first knob 206a is located at a proximal end of the handle 204 and can be used to operate the actuation assemblies 220 of the delivery apparatus 200 and the actuators 168 of the prosthetic valve 100. The first knob 206a can be configured to apply rotational movement to the actuator drivers 248. The rotation of the actuator drivers 248 can be translated to rotational motion of the actuators 168 of the prosthetic valve 100.

In an example, the second knob 206b is located at an intermediate region of the handle. The second knob 206b can be configured to release the actuation assemblies 220 from the prosthetic valve 100 (e.g., after positioning the prosthetic valve 100 at the desired implantation location and expanding the prosthetic valve 100 to the working diameter). In one example, the safety knob 202 can be configured to prevent unintentional release of the actuation assemblies 220 from the prosthetic valve. For example, the safety knob 202 can slide into a recess in the second knob 206b to prevent rotation of the second knob 206b. Retraction of the safety knob 202 from the recess can allow the second knob 206b to be rotated.

In an example, the third knob 206c is located at a distal end of the handle 204. The third knob 206c can be configured such that rotation of the knob relative to the handle body results in the outer delivery shaft 224 moving axially relative to the actuation assemblies 220, the prosthetic valve 100, and the nosecone shaft 232.

In one example, a delivery capsule 226 (shown in FIG. 3B) can be attached to a distal end of the outer delivery shaft 224. Axial movement of the outer delivery shaft 224 in a distal direction relative to the other shafts and prosthetic valve can move the delivery capsule 226 over the distal end portions of the actuation assemblies 220 and the prosthetic valve 100 (i.e., when the prosthetic valve 100 is in the radially compressed configuration) such that the prosthetic valve 100 is enclosed within the delivery capsule. Axial movement of the outer delivery shaft 224 in a proximal direction relative to the other shafts and the prosthetic valve can retract the delivery capsule 226 from the prosthetic valve 100, exposing the prosthetic valve, for example, for deployment at an implantation location.

During expansion of the prosthetic valve 100, rotation of the actuators 168 can apply moment forces to the frame 104, i.e., due to the frictional forces acting between the frame 104 and the actuator rods 172 of the actuators 168. These moment forces can, in some instances, result in the frame 104 rotating or pivoting about the longitudinal axial L of the frame during the expansion/contraction procedure. To help reduce such rotation of the entire frame, the actuators 168 can be divided into two sets, and the two sets can be rotated in opposite directions such that the moment forces due to one set of actuators is counterbalanced by the moment forces due to the other set of actuators. This can, for example, help the frame 104 to remain rotationally fixed or at least substantially rotationally fixed during expansion of the prosthetic valve. Thus, this configuration can, for example, make positioning and/or deploying a prosthetic valve relatively easier and/or predictable.

FIG. 7 illustrates a partial view of one example of a prosthetic valve 100 with a second motion transmitting member 280 attached thereto, as viewed from the inside of the valve. The second member distal end 284 can be releasably coupled to the frame 104 or a component attached to the frame, such as an inner or an outer skirt (not shown). The second motion transmitting member 280 is configured correspond to movement of the inflow end 116 of the valve. In some examples, the second member distal end 284 is releasably coupled to the prosthetic valve 100 at its inflow end 116, such that when inflow end 116 is moved along the axial or longitudinal direction, the second member distal end 284 moves therewith as long as it is attached to the valve 100.

Any reference to movement of a component in an axial direction throughout the current specification, refers to movement in a proximal or a distal direction.

In some examples, as illustrated in FIG. 7, the second member distal end 284 is releasably coupled to an inflow apex 114. Nevertheless, it is to be understood that the second member distal end 284 can be similarly attached to any other portion along a lower post member 164, including closer to nut 180, for example. Since the lower post member 164 has a constant length parallel to longitudinal axis L, which does not change during transition of the prosthetic valve 100 between compressed and expanded states thereof, axial movement of second member distal end 284 along with any portion of the lower post member 164, is directly correlative to movement of the inflow end 116.

The second member distal end 284 can be similarly attached to any other portion of the prosthetic valve 100 which moves in an axial direction along with inflow end 116. In some implementations, second member distal end 284 can be releasably coupled to any portion of a support post 124, including any portion of a cantilevered strut 148 (exemplary implementations not illustrated). Since each support post 124 has a constant length parallel to longitudinal axis L, which does not change during transition of the prosthetic valve 100 between compressed and expanded states thereof, axial movement of second member distal end 284 along with any portion of the support post 124, including cantilevered strut 148, is directly correlative to movement of the inflow end 116.

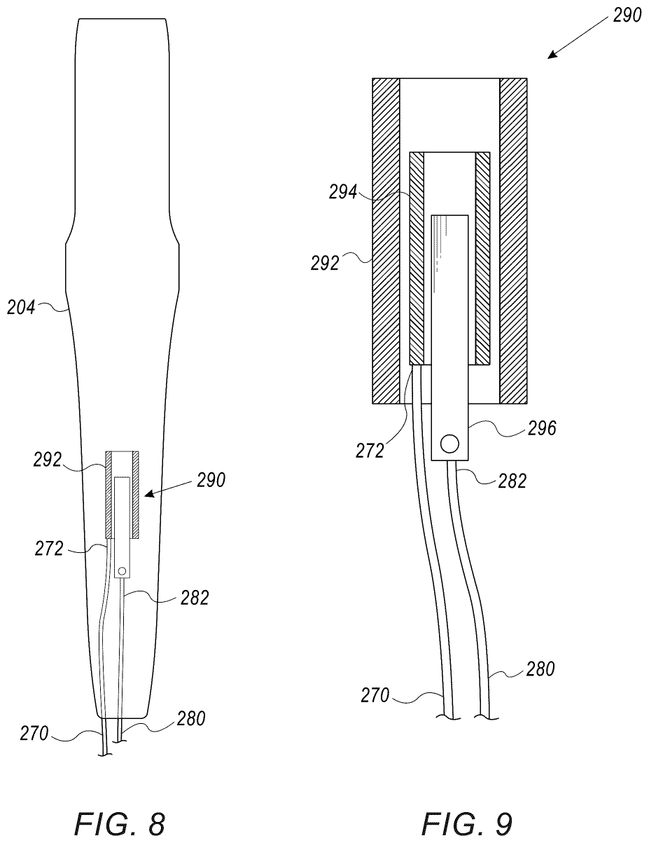

Delivery apparatus 200 can further include a measurement device 290 for measuring, in real time, the axial distance between the first member distal end 274 and the second member distal end 284. FIG. 8 schematically illustrates a measurement device 290 residing within the handle 204. Measurement device 290 can comprise a sensor 292, the first motion transmitting member 270, and the second motion transmitting member 280. FIG. 9 schematically illustrates an example of a measurement device 290 comprising a sensor 292 implemented as a linear displacement sensor. As shown in FIGS. 8-9, the first motion transmitting member 270 has a first member proximal end 272 that can be coupled to the sensor 292, and the second motion transmitting member 280 has a second member proximal end 282 that can be also coupled to the sensor 292.

The first and second motion transmitting members 270, 280 can be, for example, wires, rods, shafts, or other flexible, inelastic members configured to have sufficient rigidity such that the members do not bend, buckle, or stretch or compress axially, when a proximal or distal force is applied thereto during normal use. In some examples, the first and second motion transmitting members 270, 280 can be cylindrical in shape. Alternatively, any of the first or second motion transmitting members 270, 280 can have any various other shapes in cross-section, such as, but not limited to: square, triangular, rectangular, and the like.

In some examples, linear displacement sensor 292 is implemented as a linear variable differential transformer (LVDT) sensor, comprising a transformer core 296 within a tube 294, as illustrated in FIG. 9. In other implementations, sensor 292 can include one or more linear potentiometers. An LVDT sensor can generally have three coils (not shown) placed end to end around tube 294 in which transformed core 296, which can be a cylindrical ferromagnetic core, is disposed. The tube 294 can be attached to, for example, the first member proximal end 272. The transformed core 296 can be attached to, for example, the second member proximal end 282. Assuming that the first motion transmitting member 270 assumes a position that does not change in the axial direction, as the second member distal end 284 moves in the proximal or distal direction, relative to the first member distal end 274, the transformed core 296 moves relative to the tube 294 creating voltage differential between the coils, which can be converted by sensor 292 into relative distance between both member distal ends 274 and 284, corresponding to the change in position of the prosthetic valve's inflow end 116 (in the axial direction) relative to the position of first member distal end 274.

Although the coupling of second member proximal end 282 to transformed core 296 is illustrated as being a direct connection, this is not meant to be limiting in any way. In another example, a cable (not shown) extends from transformed core 296, and second member proximal end 282 is coupled to the cable. Similarly, although the coupling of first member proximal end 272 to tube 294 is illustrated as being a direct connection, this is not meant to be limiting in any way. In another example, a cable or any other suitable intermediate component (not shown) extends from tube 294, and first member proximal end 272 is coupled to the intermediate component.

An output of measurement device 290 can be in communication with sensor data unit 210. The measurement device 290 can be operatively coupled to a sensor data unit 210 using one or more wires or cables, or via a wireless communication link. The sensor data unit 210 can be configured to receive signals from the sensor 292 representative of the relative axial movement of the first and second member distal ends 274, 284. The sensor data unit 210 can be configured to continuously calculate the axial position of the valve's inflow end 116 relative to the position of first member distal end 274 based on the measurement inputs provided by the sensor 292. In further examples, sensor data unit 210 can be configured to output an indication of this relative position of the inflow end 116 to a user display of handle 204, such as display 212, or an external user display.

The sensor data unit 210 can comprise, in some implementations, a central processing unit (CPU), a microprocessor, a microcomputer, a programmable logic controller, an application-specific integrated circuit (ASIC) and/or a field-programmable gate array (FPGA), without limitation.

Second member distal end 284 is configured to remain attached to the prosthetic valve 100 during delivery to the implantation site, and during the implantation procedure, at least as long as determination of the position of inflow end 116 of valve 100 is required. Second member distal end 284 is further configured to detach from the prosthetic valve 100 when determination of inflow end's position is no longer required, for example—after final expansion of the prosthetic valve 100 at the site of implantation, to allow retrieval thereof along with the remainder of delivery apparatus 200 from the patient's body.

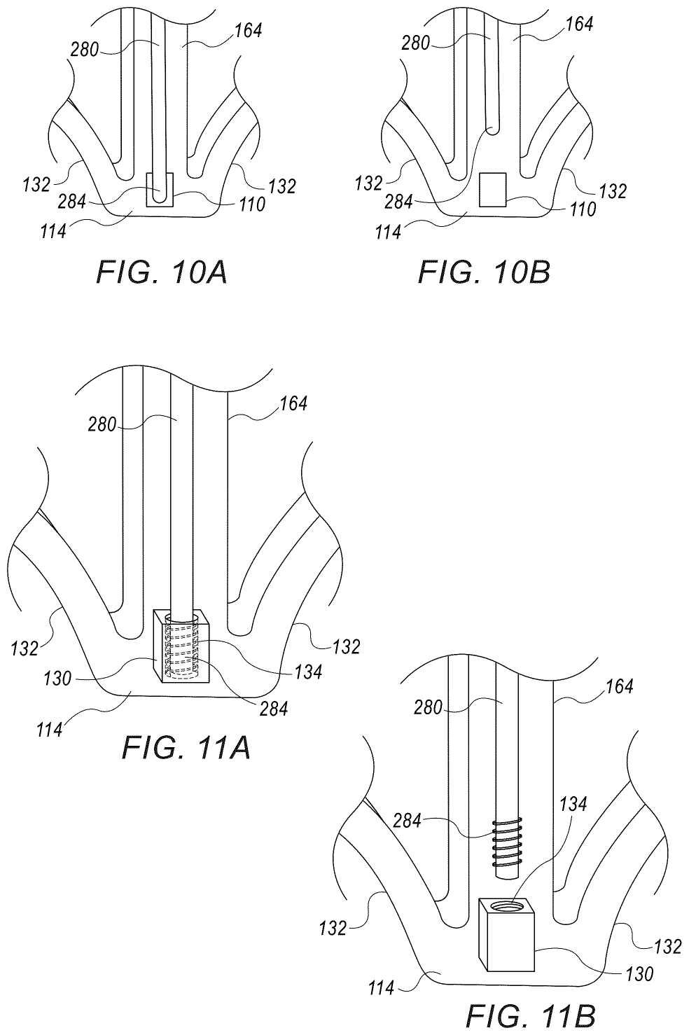

Various releasable attachment mechanisms can be implemented for second member distal end 284. FIGS. 10A-B illustrate one example of glued releasable attachment of second member distal end 284 to prosthetic valve 100. The second member distal end 284 can be glued to a component of the prosthetic valve 100, such as an inflow apex 114 as shown in FIG. 10A, or to any other portion of a lower post member 164 or a support post 124, as described above. In some implementations, an intermediate component such as patch 110 can be utilized to facilitate gluing of the second member distal end 284 to prosthetic valve 100, though this is shown merely as an option, and in other implementations, second member distal end 284 can be glued directly to prosthetic valve 100 without any additional intermediate components.

The adhesive forces of the glued attachment are set to resist spontaneous unintended detachment of the second member distal end 284 from prosthetic valve 100 during delivery through the patient's body to the implantation site, and while prosthetic valve 100 is being maneuvered during the implantation procedure. Detachment of the second member distal end 284 from prosthetic valve 100, as shown in FIG. 10B, is possible by forcibly pulling the second motion transmitting member 280 away (in a proximal direction) from valve 100, wherein the pull force is high enough to overcome the above-mentioned adhesive forces of the glued attachment.

FIGS. 11A-B illustrate another example of threaded releasable attachment of second member distal end 284 to prosthetic valve 100. In the illustrated example, the prosthetic valve 100 further comprises a coupler 130 that can be attached to the frame 104. The coupler 130 can be attached to a component of the frame 104, such as an inflow apex 114 as shown in FIG. 11A, or to any other portion of a lower post member 164 or a support post 124, as described above. The coupler 130 can includes a threaded bore 134, and the second member distal end 284 can include a complementary threading (e.g., an outer threading), configured to threadedly engage with the coupler's threaded bore 134. When detachment is desired, the second motion transmitting member 280 can be rotated around its longitudinal axis, for example via an appropriate mechanism in the handle (not shown), allowing it to be released from the coupler 130, and thereby, from prosthetic valve 100, as illustrated in FIG. 11B.

In alternative implementations, the coupled can be shaped to define an outer threading, instead of including an internal threaded bore, and the second member distal end can include an inner threaded socket configured to be threaded over the coupler.

It is to be understood that while two releasable attachment mechanisms are illustrated throughout FIGS. 10A-11B, other releasable attachment mechanisms are contemplated. For example, a snap-fit mechanism (not shown) can be implemented to attach the second member distal end 284 to prosthetic valve 100, wherein forcibly pulling the second motion transmitting member 280 can release the second member distal end 284 from its snapped position. In another example, the second member distal end 284 can be sutured to prosthetic valve 100, wherein the suture is set to tear when the second motion transmitting member 280 is forcibly pulled away from the valve 100.

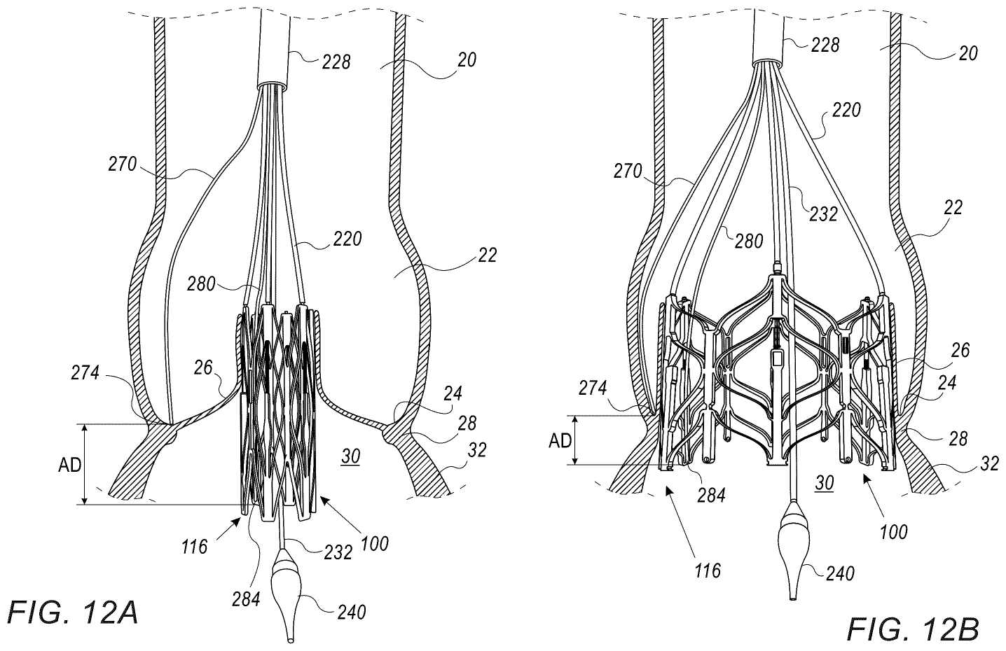

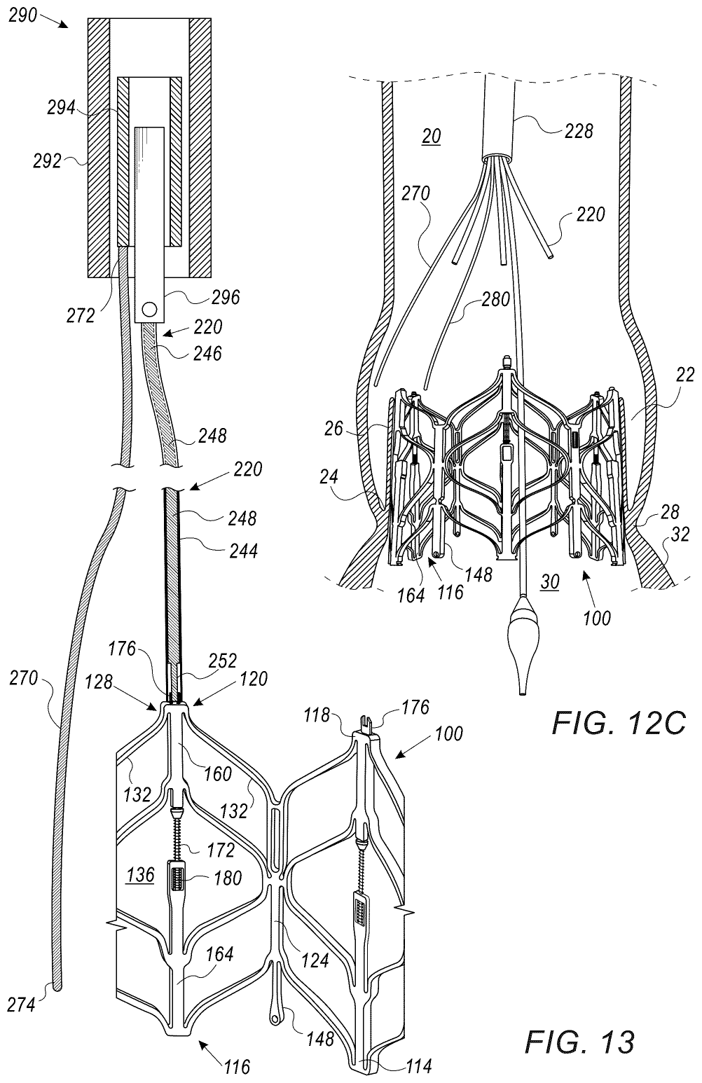

As depicted in FIGS. 12A-C, the prosthetic valve 100 can be releasably coupled to the delivery apparatus 200 during delivery, positioning and securement of the prosthetic valve 100 in a native heart valve annulus. In the depicted implantation procedure, the prosthetic valve 100 is implanted in a native aortic annulus 28 of a heart using a transfemoral delivery approach. In other examples, the prosthetic valve 100 can be implanted at other locations (e.g., a mitral valve, a tricuspid valve, and/or a pulmonary valve) and/or using other delivery approaches (e.g., transapical, transaortic, transseptal, etc.).

The prosthetic valve 100 can be releasably coupled to actuation assemblies 220 as described above, as well as to second motion transmitting member 280. A distal end of the shaft assembly 208, which comprises the radially compressed prosthetic valve 100, optionally within a delivery capsule 226, can be inserted percutaneously into a patient's vasculature and advanced toward a heart. In the illustrated example, the prosthetic valve 100 is carried in its compressed state, by delivery apparatus 200, through the aorta 20 and toward the native aortic annulus 28. When reaching the target site of implantation, the prosthetic valve can be exposed out of the outer delivery shaft 224, to be disposed in or adjacent a native aortic annulus 28. FIG. 12A shows the prosthetic valve 100 extending through the native aortic annulus 28, for example between native aortic leaflets 26, such that a portion of the prosthetic valve 100 can protrude into the left ventricle 30, optionally having its inflow end 116 disposed within the left ventricle outflow tract (LVOT) 32.

As further shown in FIG. 12A, the distal portion of the first motion transmitting member 270, which includes the first member distal end 274, is also exposed at this stage, prior to valve expansion, and is distally advanced alongside the prosthetic valve 100 until the first member distal end 274 encounters the floor 24 of the aortic sinuses 22, and cannot be advanced any further.

In some examples, a portion of the first motion transmitting member 270, such as its distal portion leading to the first member distal end 274, is pre-shaped to extend radially away from the prosthetic valve 100 once it is exposed out of the shaft assembly 208/For example, the first motion transmitting member 270 can be made of a shape memory material, with the distal portion being pre-formed or “heat-set” into a desired outwardly curved configuration. In such cases, as the distal portion of the first motion transmitting member 270 is exposed out of a shaft, such as an outer delivery shaft 224 and/or a multi-lumen delivery shaft 228, in the vicinity of the site of implantation, it will be biased sideways, radially away from the longitudinal axis L of prosthetic valve 100, facilitating easier advancement thereof toward any one of the aortic sinuses 22, until the first member distal end 274 engages the sinus floor 24 as shown in FIG. 12A.

Once the first member distal end 274 engages the sinus floor 24, no further axial movement is applied to the first motion transmitting member 270, while the prosthetic valve 100 can be still translated in a proximal or distal direction, and can be either expanded or re-compressed by the actuation assemblies 220 coupled to actuator 168. Since the second motion transmitting member 280 is coupled to the prosthetic valve 100, any movement of the second member distal end 284, relative to the position of the first member distal end 274 at sinus floor 24, is detected by the sensor 292 of measurement device 290. Thus, the distance AD marked in FIG. 12A, designating the axial distance between the first member distal end 274 and the second member distal end 284, is continuously measured in real-time by the sensor 292. By extension, the distance between the valve's inflow end 116 and the plane of the sinus floor 24 is continuously monitored in real-time.

Any axial movement of prosthetic valve 100, drags the second member distal end 284, which is coupled thereto, there-along in the same direction. If the second member distal end 284 is attached to the prosthetic valve at its inflow end 116, such as to an inflow apex 114, the axial distance AD between both member distal ends 274, 284 is substantially equal to the distance between the plane of the sinus floor 24 and the valve's inflow end 116. In other cases, as mentioned above, the second member distal end 284 can be attached to the prosthetic valve 100 at a position which is not precisely at the level of the inflow end 116, but it rather distanced therefrom along the axial direction by a known constant distance, such that movement of the inflow end 116 is still translated to similar linear movement of the second member distal end 284. In such cases, sensor data unit 210 can calculate the distance between the plane of the sinus floor 24 and the valve's inflow end 116, by adding the known axial distance between the second member distal end 284 and the inflow end 116, to the measured axial distance AD between both member distal ends 274, 284.

FIG. 12B shows the prosthetic valve 100 being radially expanded, for example by actuating the plurality of actuator 168 by corresponding actuation assemblies 220, as described above. As the valve expands radially, it foreshortens axially, which can result in the inflow end 116 translating in the axial direction, relative to the valve's pre-expanded or compressed state. Valve axial foreshortening can result in the inflow end 116 moving closer to the native annulus 28 during expansion, resulting in a corresponding decrease of the axial distance AD between both member distal ends 274, 284, which is correlative to a proportional decrease of the distance between the sinus floor 24 and the valve's inflow end 116, as shown in FIG. 12B, compared to the evidently greater AD of FIG. 12A.

A mechanically-expandable valve, such as prosthetic valve 100, can be either expanded or re-compressed by the actuation assemblies 220 coupled to actuators 168. For example, actuator drivers 248 can be rotated in one rotational direction to facilitate valve expansion, and in an opposite rotation direction to facilitate valve compression. Since prosthetic valve 100 can transition back-and-forth between various diameters, the position of its inflow end 116 can vary accordingly. Since the first member distal end 274 remains in a fixed position, engaged with the sinus floor 24, and the second member distal end 284 is coupled to the prosthetic valve 100 and is movable along with the inflow end 116, measurement device 290 can be utilized to continuously monitor the axial distance between the valve's inflow end 116 and the plane of the sinus floor 24 in real-time, detecting any changes in position of the inflow end 116 responsive to such maneuvering of the prosthetic valve 100. The measured distance can be optionally displayed to the operator of the delivery apparatus 200 on a display 212 at the handle 204, and/or transmitted, via wired or wireless communication, to an external device, including being displayed on an external monitor.

In some cases, the distance between inflow end 116 and the plane of the sinus floor 24, based on the axial distance AD between both member distal ends 274, 284, can be compared to pre-stored maximal and/or minimal threshold values. For example, an axial distance AD that is too high, exceeding a maximal threshold value, may be indicative of the prosthetic valve positioned too low relative to the native annulus. If an prosthetic valve protrudes too deep into the LVOT 32, it may result in hemodynamic disturbances, and may inflict temporary or permanent injury to the conduction system. Similarly, an axial distance AD that is smaller than a minimal threshold value, may be indicative of the prosthetic valve positioned too high relative to the native annulus, posing a potential risk of the prosthetic valve dislodging from its location during or after implantation. Sensor data unit 210 can compare measured distances to pre-stored threshold values, and optionally provide visual and/or auditory feedback when the measured distance exceed such thresholds.

Once the prosthetic valve 100 is expanded to its functional diameter and secured within the native annulus 28, the actuation assemblies 220 and second motion transmitting member 280 are released from the valve 100 as described herein above. The delivery apparatus 200, along with both motion transmitting members 270, 280, can then be withdrawn from the patient's vasculature, as shown in FIG. 12C, leaving the prosthetic valve 100 within the native aortic annulus 28 to regulate blood flow from the left ventricle 30 into the aorta 20.

FIG. 13 shows another example, in which delivery apparatus 200 does not include a separate second motion transmitting member, but rather takes advantage of one of the actuator drivers 248 which can serve as a replacement for second motion transmitting member. In the illustrated example, one actuator driver 248, disposed within an outer sleeve 244 and attached at its distal central protrusion 252 to a corresponding actuator head 176 in the same manner described above. However, unlike any other actuator driver, the specific actuator driver 248 illustrated in FIG. 13 is coupled, at a driver proximal end 246 thereof, to the transformer core 296, in the same manner described above for attachment of second member proximal end (282) to the core (296).

A measurement device 290 that includes a first motion transmitting member 270 and an actuator driver 248 serving as the second motion transmitting member, can be employed in a similar manner to that described for measurement device provided with two motion transmitting members 270, 280, with the exception that the measured axial distance AD between the first member distal end 274 and the central protrusion 252 is not directly indicative of the distance between first member distal end 274 and the inflow end 116, but rather needs to account for the fact that the central protrusion 252 of actuator driver 248 is attached to the upper actuator head 176 of the corresponding actuator 172, and therefore moves along with the outflow end 120 instead of the inflow end 116. This can be accounted for due to the fact that the number of rotations of the actuator driver 248, and consequently, of the actuator 168 coupled thereto, is directly correlative to the distance between the upper post member 160 and the lower post member 164, and by extensions, to the distance between the outflow end 120 and the inflow end 116 of the valve.

As long as the prosthetic valve 100 remains in a specific diameter, such that the actuator driver 248 is not rotated in either direction, the gap G does not change, meaning that the distance between the outflow end 120 and the inflow end 116 remains constant, substantially equal to the combined known lengths of upper and lower post members 160, 164 and gap G. In such states, in which the prosthetic valve 100 is not expanded to compressed, any movement of the valve in the axial direction, moves the actuator driver therewith. The axial distance AD is now measured between the first member distal end 274 and the central protrusion 252 of actuator driver 248, which can be correlative to the distance between the sinus floor 24 and the outflow end 120 of the valve. However, for a known distance between the outflow end 120 and the inflow end 116 at a specific diameter, the sensor data unit 210 can account for this distance between outflow and inflow ends 120, 116 to derive the distance between the sinus floor 24 and the inflow end 116.

As the actuator driver 248 and the actuator 172 are simultaneously rotated to expand or compress the valve 100, the gap G will decrease or increase, as explained herein above, meaning that the distance between outflow and inflow ends 120, 116 will also decrease or increase, respectively. Since the increase or decrease of the gap G is proportional to the number of rotations of the actuator driver 248 and actuator 172, the extent to which gap G is increased or decreased can be estimated by counting the number and of rotations in the relevant direction. In some example, measurement device 290 also includes a counter (not shown) configured to count the number of rotations of the actuator driver 248. The number of rotations is correlative to the absolute value of the axial translation of upper and lower post members 160, 164 relative to each other, and the direction of the rotations is indicative of whether the gap G increases or decreases, which is in turn indicative of the increased or decreased distance between outflow and inflow ends 120, 116.

In some examples, measurement device can include more than one sensor, such as a linear displacement sensor 292 that can be implemented as a linear variable differential transformer (LVDT) sensor, and another sensor for counting the number of rotations (and optionally, detecting the rotational movement direction) of actuator driver 248. The combined signals can be delivered to sensor data unit 210, which will account for both linear displacement and rotational movement of actuator driver 248 to provide an indication of the distance between the sinus floor 24 and the inflow end 116, and optionally compare this distance to pre-stored threshold values, as described hereinabove.

While FIG. 13 illustrated a configuration of an actuator driver 248 utilized as a second motion transmitting member, coupled at a proximal end 246 thereof to the sensor 292, it is to be understood that in alternative configurations, a separate motion transmitting member, such as second motion transmitting member 280 of the type described hereinabove, can be attached to an actuator driver 248 or another component of an actuation assembly 220 instead of to the prosthetic valve, and utilized in the same manner described above with respect to FIG. 13, except that the second member proximal end 282 is coupled to the sensor 292 (such as to a transformed core 296 in the case of an LVDT sensor), and the second member distal end 284 can be coupled to one of the plurality of actuation assemblies 220, such as to an actuator driver 248 (for example, coupled to its central protrusion 252) or to an outer sleeve 244 (for example, to a distal end portion of the outer sleeve, adjacent outflow end 120).

A second motion transmitting member 280 which is attached at its proximal end 282 to the sensor 292, and at its distal end 284 to an actuation assembly 220, can be utilized in the same manner described for the actuator driver 248 with respect to FIG. 13, for example, and may be advantageous over the configuration described above with respect to FIGS. 7-11B, in that the second member distal end 284 can be permanently attached to the actuation assembly, without the need to incorporate releasable attachment mechanisms.

While a procedure of aortic valve replacement, during which the first member distal end 274 can engage the sinus floor 24, is described above, it is to be understood that the same principles can be adapted for implantation procedures in other valves, and that first member distal end 274 can similarly engage any other appropriate anatomical structure which can be generally indicative of the native valve's level.

While described for use with a mechanically-expandable prosthetic valve 100 as illustrated in FIGS. 1A-B, it is to be understood that a delivery assembly equipped with a measurement device 290 can be similarly employed with other types of mechanically-expandable valves. For example, a measurement device 290 provided with first and second motion transmitting members 270, 280, or with a first motion transmitting member 270 and a rotatable driver coupled to the measurement device, can be used with mechanically-expandable prosthetic valves of the types disclosed in U.S. Pat. No. 10,603,165 and U.S. Publication Nos. 2019/0060057, and 2018/0325665, each of which is incorporated herein by reference in its entirety. Moreover, a measurement device 290 provided with first and second motion transmitting members 270, 280 can be similarly employed balloon-expandable valves or self-expandable valves, for example by attaching the second member distal end 284 to an inflow apex of such valves, or to a component that is proportionally movable along with the inflow end of such valves.

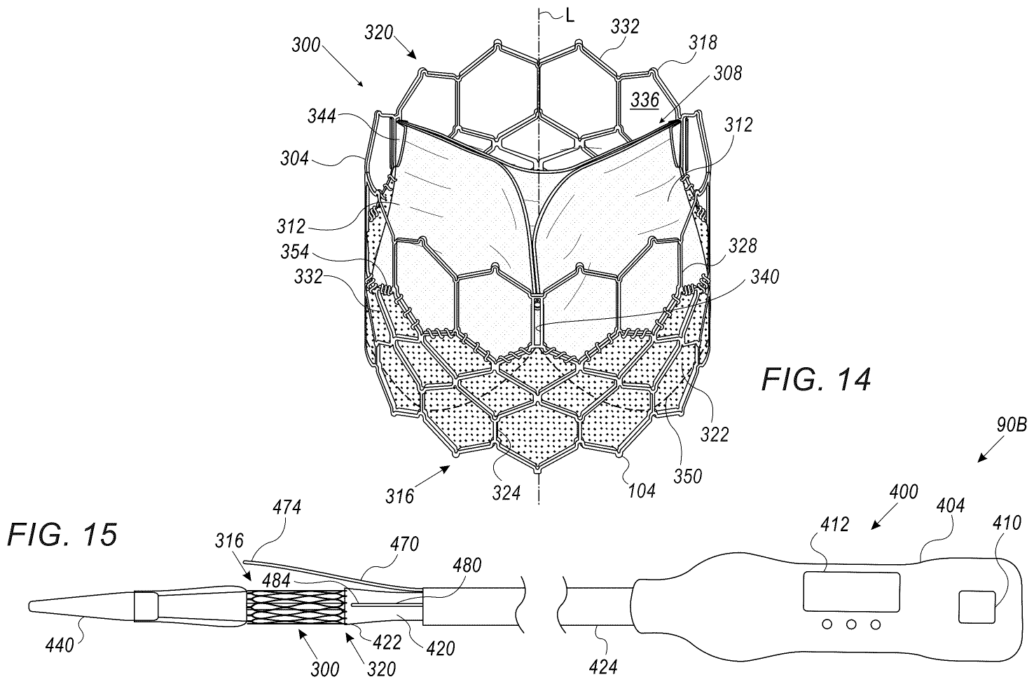

FIG. 14 illustrates a prosthetic valve 300, according to another example. The prosthetic valve 300 can be configured to replace a native heart valve (e.g., aortic, mitral, pulmonary, and/or tricuspid valves). The prosthetic valve 300 is illustrated as a balloon expandable prosthetic valve movable between a radially compressed state and a radially expanded state. Balloon expandable valves generally involve a procedure of inflating a balloon within a prosthetic valve, thereby expanding the prosthetic valve 300 within the desired implantation site. The prosthetic valve 300 can include a frame 304 having an annular shape. The prosthetic valve 300 can further include a valvular structure 308 supported within and coupled to the frame 304. The valvular structure 308 can include one or more leaflets 312, which can be identical to any example described above with respect to valvular structure 108 and leaflets 112, a detailed description of which is not repeated here in the interest of brevity.

As further illustrated in FIG. 14, the frame 304 has an inflow end 316, an outflow end 320, and a longitudinal axis L extending in a direction from the inflow end 316 to the outflow end 320. The frame 304 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 304 can be crimped to a radially compressed state on a balloon catheter 452 (shown in FIG. 15), and then expanded inside a patient by an inflatable balloon. Alternatively or additionally, the frame 304 can be made of shape-memory materials such as, but not limited to, nickel titanium alloy (e.g., Nitinol).

Frame 304 comprises a plurality of intersecting struts, including angled struts 332, and can optionally include lower vertical struts, such as lower vertical struts 324 and upper vertical struts 328. The frame 304 includes a plurality of strut rungs that can collectively define one or more rows of cells 336. The frame 304 can have a cylindrical or substantially cylindrical shape having a constant diameter from the inflow end 316 to the outflow end 320 as shown, or the frame can vary in diameter along longitudinal axis L, as disclosed in U.S. Pat. No. 9,155,619, which is incorporated herein by reference.

The end portions of angled struts 332 are forming outflow apices 318 at the outflow end 320 and inflow apices 314 at the inflow end 316. The struts can intersect at additional junctions formed between the outflow apices 318 and the inflow apices 314. The junctions can be equally or unequally spaced apart from each other, and/or from the apices 318, 314, between the outflow end 320 and the inflow end 316.

As further shown in FIG. 14, an lower rung of cells 336 can include a plurality of lower vertical struts 324, and an upper rung of cells 336 can include a plurality of upper vertical struts, some of which can include commissure windows 340 that can accommodate commissures 344 of the valvular structure 308. However, it is to be understood that in alternative configurations, the frame does not necessarily include vertical struts, or can include vertical struts disposed elsewhere between angled struts 332. Similarly, a frame 304 is not limited to the number of rungs of cells illustrated in FIG. 14, but may rather include anywhere between a single rung of cells and any plurality of rungs of cells, wherein each rung of cells can include any number of cells disposed around the circumference of the valve.

Prosthetic valve 300 can further include an inner skirt 322 as illustrated in FIG. 14 and/or an outer skirt (not shown), which can be implemented according to any example described for prosthetic valve 100, a detailed description of which is not repeated here in the interest of brevity. As shown, the inner skirt 322 can be attached to the frame 304 (e.g., using sutures 354), and lower edges of the leaflets 312 can be sutured to the inner skirt 322 along a scalloped line 350.

FIG. 15 illustrate a delivery assembly 90B, which can includes a prosthetic valve 100 and a delivery apparatus 400, according to one example. The delivery apparatus 400 can be used to deliver the prosthetic valve 300 to an implantation location within a patient's body. The delivery apparatus 400 includes a handle 404 and a balloon catheter 452 having an inflatable balloon 450 mounted on its distal end. The balloon-expandable prosthetic valve 300 can be carried in a crimped state over the balloon catheter 452. Optionally, an outer delivery shaft 424 can concentrically extend over the balloon catheter 452, and a push shaft 420 disposed over the balloon catheter 452, optionally between the balloon catheter 452 and the outer delivery shaft 424, having a push shaft distal end portion 422 opposite to the handle 404.

The outer delivery shaft 424, the push shaft 420, and the balloon catheter 452, can be configured to be axially movable relative to each other. For example, a proximally oriented movement of the outer delivery shaft 424 relative to the balloon catheter 452, or a distally oriented movement of the balloon catheter 452 relative to the outer delivery shaft 424, can expose the prosthetic valve 300 from the outer delivery shaft 424. The delivery apparatus 400 can further include a nosecone 440 carried by a nosecone shaft 432 extending through a lumen of the balloon catheter 452. The nosecone 440 and nosecone shaft 432 can be identical to nosecone 240 and nosecone shaft 232 described for delivery apparatus 200, a detailed description of which is not repeated here in the interest of brevity.

The proximal ends of the balloon catheter 452, the outer delivery shaft 424, the push shaft 420, and optionally the nosecone shaft 432, can be coupled to the handle 404. During delivery of the prosthetic valve 300, the handle 404 can be maneuvered by an operator (e.g., a clinician or a surgeon) to axially advance or retract components of the delivery apparatus 400, such as the nosecone shaft 432, the balloon catheter 452, the outer delivery shaft 424, and/or the push shaft 420, through the patient's vasculature, as well as to inflate the balloon 450 mounted on the balloon catheter 452, so as to expand the prosthetic valve 300, and to deflate the balloon 450 and retract the delivery apparatus 400 once the prosthetic valve 300 is mounted in the implantation site.

In addition, the delivery apparatus 400 can optionally include a sensor data unit 410, and one or more visual or auditory informative elements configured to provide visual or auditory information and/or feedback to a user or operator of delivery apparatus 400, such as a display 412, LED lights, speakers (not shown) and the like.

Delivery apparatus 400 can further include a first motion transmitting member 470 extending distally from the handle 404 and terminating at a member distal end 474, and a second motion transmitting member 480 extending distally from the handle 404 and terminating at a second member distal end 484. First member distal end 474 is a free end, not attached to any components of the delivery apparatus 400 or prosthetic valve 300, while second member distal end 484 is attached to the push shaft 420, and in some implementations, to the push shaft distal end portion 422. Any of the first motion transmitting member 470 and/or second motion transmitting member 480 can extend through the outer delivery shaft 424, for example between the inner surface of the outer delivery shaft 424 and the outer surface of the push shaft 420.

The first motion transmitting member 470 can be concealed within outer delivery shaft 424 during delivery, and can have its distal end exposed when reaching closer to the site of implantation, for example by extending distally from outer delivery shaft 424, while its first member distal end 474 is free-ended and is not attached to the prosthetic valve 300 or any other shaft of the delivery apparatus 400. Moreover, when the first member distal end 474 is axially positioned between the level of the outflow end 320 and the inflow end 316, it is disposed out of the prosthetic valve 300, away from longitudinal axis L.

It is to be understood that delivery apparatus 200 can be provided, in some implementations, without optional outer delivery shaft 424, nosecone 440 and nosecone shaft 432, sensor data unit 410, and/or display 412, without exceeding the scope of the disclosure.

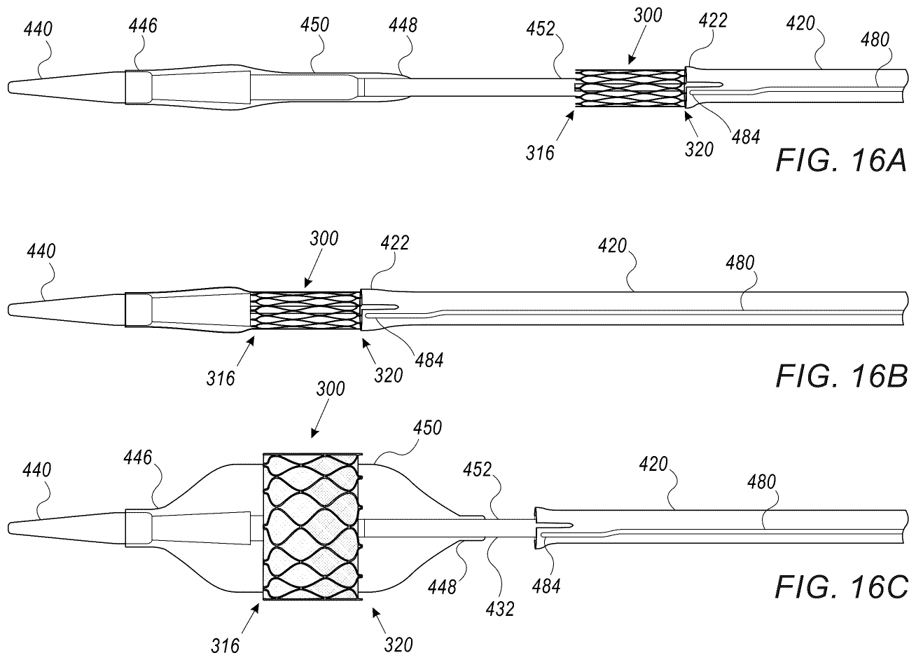

As depicted in FIGS. 16A-C, the prosthetic valve 300 can be carried by the delivery apparatus 400 during delivery in a crimped state, and expanded by balloon inflation to secure it in a native heart valve annulus. In the depicted implantation procedure, the prosthetic valve 300 is initially crimped over the balloon catheter, proximal to the inflatable balloon 450. Because prosthetic valve 300 is crimped at a location different from the location of balloon 450, prosthetic valve 300 can be crimped to a lower profile than would be possible if it was crimped on top of balloon 450. This lower profile permits the clinician to more easily navigate the delivery apparatus 400 (including crimped prosthetic valve 300) through a patient's vasculature to the treatment location. The lower profile of the crimped prosthetic valve is particularly helpful when navigating through portions of the patient's vasculature which are particularly narrow, such as the iliac artery.

As shown in FIG. 16A, the balloon 450 can be secured to balloon catheter 452 at its balloon proximal end 448, and to either the balloon catheter 452 or the nosecone 440 at its balloon distal end 446. The push shaft distal end portion 422 is positioned proximal to the outflow end 320 of the prosthetic valve 300.

When reaching the site of implantation, and prior to balloon inflation, the push shaft 420 is advanced distally, allowing the distal edge of its distal end portion 422 to contact and push against the outflow end 320 of prosthetic valve 300, pushing the valve 300 distally therewith. The push shaft distal end portion 422 is dimensioned to engage with the outflow end 320 of the prosthetic valve 300 in a crimped configuration of the valve. In some implementations, the push shaft distal end portion 422 can be flared radially outward, to terminate at a wider-diameter distal edge that can contact the prosthetic valve 300 in its crimped state. Push shaft 420 is advanced distally, pushing the prosthetic valve 300 therewith, until the crimped prosthetic valve 300 is disposed around the balloon 450, as shown in FIG. 16B, at which point the balloon can be inflated to radially expand the prosthetic valve 300, as shown in FIG. 16C. Once the prosthetic valve 300 is expanded to its functional diameter within a native annulus, the balloon 450 can be deflated, and the delivery apparatus 400 can be retrieved from the patient's body.

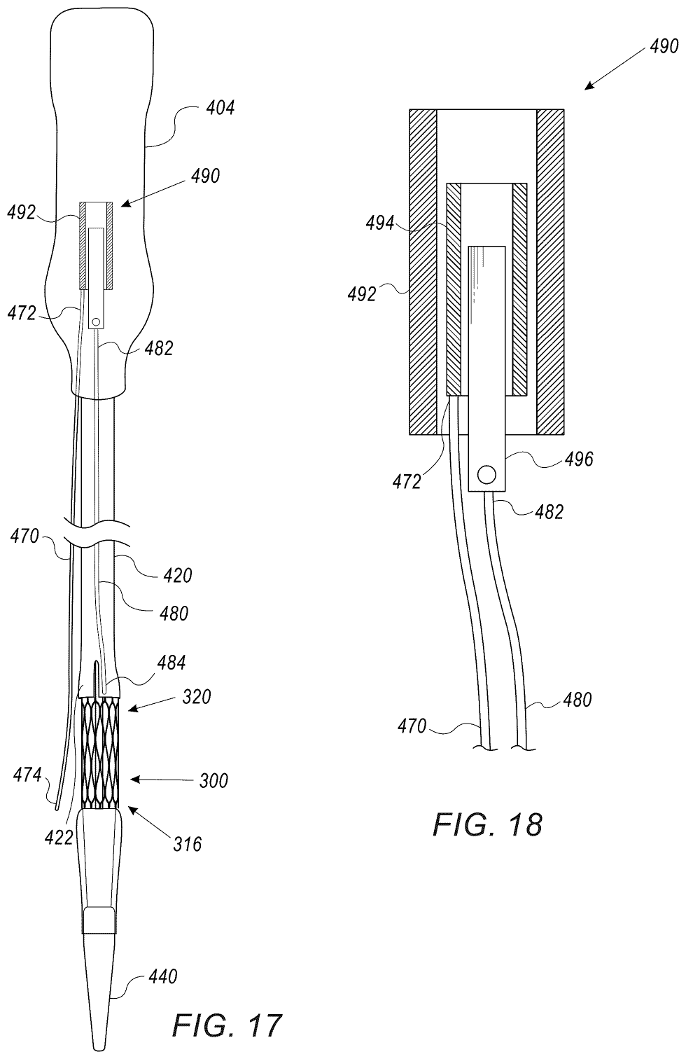

Delivery apparatus 400 can further include a measurement device 490 for measuring, in real time, the axial distance between the first member distal end 474 and the second member distal end 484. FIG. 17 schematically illustrates a measurement device 490 residing within the handle 404. Measurement device 490 can comprise a sensor 492, the first motion transmitting member 470, and the second motion transmitting member 480. FIG. 18 schematically illustrates an example of a measurement device 490 comprising a sensor 492 implemented as a linear displacement sensor. As shown in FIGS. 17-18, the first motion transmitting member 470 has a first member proximal end 472 that can be coupled to the sensor 492, and the second motion transmitting member 480 has a second member proximal end 482 that can be also coupled to the sensor 492.

The first and second motion transmitting members 470, 480 can be, for example, wires, rods, shafts, or other flexible, inelastic members configured to have sufficient rigidity such that the members do not bend, buckle, or stretch or compress axially, when a proximal or distal force is applied thereto during normal use. In some examples, the first and second motion transmitting members 470, 480 can be cylindrical in shape. Alternatively, any of the first or second motion transmitting members 470, 480 can have any various other shapes in cross-section, such as, but not limited to: square, triangular, rectangular, and the like.

In some examples, linear displacement sensor 492 is implemented as an LVDT sensor, comprising a transformer core 496 within a tube 494, as illustrated in FIG. 18. In other implementations, sensor 492 can include one or more linear potentiometers. An LVDT sensor can generally have three coils (not shown) placed end to end around tube 494 in which transformed core 496, which can be a cylindrical ferromagnetic core, is disposed. The tube 494 can be attached to, for example, the first member proximal end 472. The transformed core 496 can be attached to, for example, the second member proximal end 482. Assuming that the first motion transmitting member 470 assumes a position that does not change in the axial direction, as the second member distal end 484 moves in the proximal or distal direction, relative to the first member distal end 474, the transformed core 496 moves relative to the tube 494 creating voltage differential between the coils, which can be converted by sensor 492 into relative distance between both member distal ends 474 and 484, corresponding to the change in position of the prosthetic push shaft distal end portion 422 (in the axial direction) relative to the position of first member distal end 474, which is, in turn, indicative to the change in position of the valve's outflow end 320 and the valve's inflow end 316 when the push shaft distal end portion 422 abuts outflow end 320, prior to balloon inflation.

Although the coupling of second member proximal end 482 to transformer core 496 is illustrated as being a direct connection, this is not meant to be limiting in any way. In another example, a cable (not shown) extends from transformer core 496, and second member proximal end 482 is coupled to the cable. Similarly, although the coupling of first member proximal end 472 to tube 494 is illustrated as being a direct connection, this is not meant to be limiting in any way. In another example, a cable or any other suitable intermediate component (not shown) extends from tube 494, and first member proximal end 472 is coupled to the intermediate component.

An output of measurement device 490 can be in communication with sensor data unit 410. The measurement device 490 can be operatively coupled to a sensor data unit 410 using one or more wires or cables, or via a wireless communication link. The sensor data unit 410 can be configured to receive signals from the sensor 492 representative of the relative axial movement of the first and second member distal ends 474, 484. The sensor data unit 410 can be configured to continuously calculate the axial position of the valve's inflow end 316 relative to the position of first member distal end 474 based on the measurement inputs provided by the sensor 492. In further examples, sensor data unit 410 can be configured to output an indication of this relative position of the inflow end 316 to a user display of handle 404, such as display 412, or an external user display.

The sensor data unit 410 can comprise, in some implementations, a central processing unit (CPU), a microprocessor, a microcomputer, a programmable logic controller, an application-specific integrated circuit (ASIC) and/or a field-programmable gate array (FPGA), without limitation.

As shown in FIG. 16A-B, the push shaft distal end portion 422 abuts outflow end 320 of prosthetic valve 300 during prosthetic valve positioning within the native annulus, prior to expansion thereof by balloon inflation. This means that the position of push shaft distal end portion 422 is correlative to the position of outflow end 320. As long as the prosthetic valve 300 remains in a crimped state, its axial length retains a constant, known value, meaning that the position of the push shaft distal end portion 422 is also correlative to the position of inflow end 316. Thus, delivery apparatus 400 carrying prosthetic valve 300 can be utilized in a substantially similar manner to that described above with respect to FIGS. 12A-C for the delivery apparatus 200 carrying prosthetic valve 100, meaning that the delivery apparatus 400 can be utilized to similarly deliver prosthetic valve 300 to an implantation site, such as the native aortic annulus 28, at which point the distal portion of the first motion transmitting member 470 can be exposed and advanced toward the aortic sinus 22 until the first member distal end 474 engages the sinus floor 24.

The prosthetic valve 300 is advanced by the push shaft 420 until it is disposed, still in a crimped state thereof, over the balloon 450. The second member distal end 484 is attached to the push shaft 420, such that the position of the inflow end 316 of the prosthetic valve 300, which is still in its crimped state, can be extrapolated from the position of the first member distal end 474, with respect to the position of the first member distal end 474. Assuming that a correlation between inflation of balloon 450 and the resulting expanded diameter of prosthetic valve 300 is known, and that the extent to which prosthetic valve 300 foreshortens for each expanded diameter thereof is also known, inflation of balloon 450 to expand the prosthetic valve 300 to any specific diameter will result in a known change of the distance between the outflow end 320 and the inflow end 316, meaning that the distance of inflow end 316 in an expanded state of prosthetic valve 300, relative to first member distal end 474, can be calculated, for example by sensor data unit 410, on the basis of the measured distance of second member distal end 484 (indicative of the position of outflow end 320 in an expanded state of the valve 300 as well), and a known foreshortening of the valve 300 in an expanded diameter thereof.

In some examples, the second member distal end 484 is permanently coupled to the push shaft 420. The term “permanently coupled”, as used herein, refers to two components, such as the second member distal end 484 and the push shaft 420, being coupled in such a way that the two components cannot be separated without plastically deforming at least one of the components.

Unlike mechanically expandable prosthetic valve 100, a conventional balloon-expandable prosthetic valve 300 is not necessarily re-compressible after expansion, meaning that based on the position of second member distal end 484 adjacent outflow end 320, and known or estimated foreshortening of the prosthetic valve 300 when expanded by balloon inflation, it may be sufficient to predict or estimate the distance between inflow end 316 of the valve in its expanded state, and the plane of the sinus floor 24, based on the measured axial distance AD between the second member distal end 484, attached to the push shaft 420, and the first member distal end 474 engaged with sinus floor 24. This configuration can be advantageous over direct attachment of a distal end 284 of a second motion transmitting member 280 to the prosthetic valve (such as to prosthetic valve 100), since no releasable attachment mechanism is required for delivery apparatus 400, and the second member distal end 484 can be permanently attached to the push shaft 420, allowing it to be retrieved along with the push shaft 420 and the remainder of the delivery apparatus 400 from the patient's body, without requiring any additional steps for decoupling the second motion transmitting member. This in turn simplifies both production of the delivery apparatus 400, and the procedural complexity of the implantation and delivery apparatus retrieval process.

While second member distal end 484 is illustrated attached to the distal edge of the push shaft, it is to be understood that in other implementations, the second member distal end 484 can be attached to a different, more proximal position, relative to the distal edge of the push shaft, assuming that the distance between the point of attachment of the second member distal end 484 to the distal edge of the push shaft, and thus, to the inflow end of the valve, is known and does not change during the implantation procedure.

While a delivery apparatus with a non-detachable measurement device, such as delivery apparatus 400, is illustrated for use in combination with a balloon-expandable prosthetic valve 300, and a delivery apparatus with a detachable measurement device, such as delivery apparatus 200, is illustrated for use in combination with a mechanically-operable prosthetic valve 100, it is to be understood that a delivery apparatus with a detachable measurement device, such as a delivery apparatus 200, can be similarly utilized, in examples, with a balloon-expandable prosthetic valve 300, wherein the first member distal end 274 can be releasably coupled to the inflow end 316 of the prosthetic valve 300, for example in the same manner described above with respect to FIGS. 10A-11B, mutatis mutandis.

### Some Examples of the Disclosed Technology

Some examples of above-described implementations are enumerated below. It should be noted that one feature of an example in isolation or more than one feature of the example taken in combination and, optionally, in combination with one or more features of one or more examples below are examples also falling within the disclosure of this application.
Example 1. A delivery assembly, comprising:
a prosthetic valve having an inflow end and an outflow end, the prosthetic valve comprising a frame movable between a radially compressed and a radially expanded configuration; and
a delivery apparatus comprising:
   a handle;
   a measurement device comprising:
      a sensor residing within the handle;
      a first motion transmitting member coupled at a first member proximal end thereof to the sensor, and extending from the handle to an opposite first member distal end; and
      a second motion transmitting member coupled at a second member proximal end thereof to the sensor, and extending from the handle to an opposite second member distal end;
wherein the first member distal end is a free end;
wherein the second member distal end is releasably coupled to the prosthetic valve; and
wherein the sensor is configured to sense, in real time, axial movement of the second
member distal end relative to the first member distal end.
Example 2. The delivery assembly of any example herein, particularly example 1, wherein the sensor is a linear displacement sensor.
Example 3. The delivery assembly of any example herein, particularly example 2, wherein the linear displacement sensor comprises a linear variable differential transformer (LVDT) sensor.
Example 4. The delivery assembly of any example herein, particularly example 3, wherein the LVDT sensor comprises a tube and a transducer core axially movable within the tube, wherein the first member proximal end is attached to the tube, and wherein the second member proximal end is attached to the transducer core.
Example 5. The delivery assembly of any example herein, particularly any one of examples 1 to 4, wherein the delivery apparatus further comprises a shaft assembly comprising an outer delivery shaft, wherein the first motion transmitting member and the second motion transmitting member extend through the outer delivery shaft.
Example 6. The delivery assembly of any example herein, particularly example 5, wherein the shaft assembly further comprises a multi-lumen delivery shaft disposed within the outer delivery shaft, and wherein at least one of the first motion transmitting member or the second motion transmitting member extends through the multi-lumen delivery shaft.
Example 7. The delivery assembly of any example herein, particularly example 6, wherein both the first motion transmitting member and the second motion transmitting member extend through corresponding separate lumens of the multi-lumen delivery shaft.
Example 8. The delivery assembly of any example herein, particularly example 6, wherein the first motion transmitting member extends through the multi-lumen delivery shaft, and the second motion transmitting member extends through the outer delivery shaft, between an outer surface of the multi-lumen delivery shaft and an inner surface of the outer delivery shaft.
Example 9. The delivery assembly of any example herein, particularly any one of examples 1 to 8, wherein the second member distal end is releasably coupled to the inflow end of the prosthetic valve.
Example 10. The delivery assembly of any example herein, particularly example 9, wherein the second member distal end is releasably coupled to an inflow apex of the prosthetic valve.
Example 11. The delivery assembly of any example herein, particularly any one of examples 1 to 10, wherein the second member distal end is releasably coupled to the frame.
Example 12. The delivery assembly of any example herein, particularly example 11, wherein the frame comprises a plurality of support posts aligned with a longitudinal axis of the prosthetic valve, and wherein the second member distal end is releasably coupled to one of the support posts.
Example 13. The delivery assembly of any example herein, particularly example 12, wherein the support post to which the second member distal end is attached, further comprises a commissure window.
Example 14. The delivery assembly of any example herein, particularly example 12, wherein the support post to which the second member distal end is attached, further comprises a cantilevered strut extending toward the inflow end.
Example 15. The delivery assembly of any example herein, particularly example 12, wherein at least one of the support posts comprises an upper post member and a lower post member, and wherein the second member distal end is releasably coupled to the lower post member.
Example 16. The delivery assembly of any example herein, particularly any one of examples 1 to 15, wherein the second member distal end is glued to the prosthetic valve.
Example 17. The delivery assembly of any example herein, particularly example 16, further comprising a patch disposed between the second member distal end and the prosthetic valve.
Example 18. The delivery assembly of any example herein, particularly any one of examples 1 to 15, wherein the prosthetic valve further comprises a coupler attached to the frame, and wherein the second member distal end is threadedly engaged with the coupler.
Example 19. The delivery assembly of any example herein, particularly example 18, wherein the coupler comprises a threaded bore, and wherein the second member distal end comprises an outer threading.
Example 20. The delivery assembly of any example herein, particularly any one of examples 1 to 19, wherein the delivery apparatus further comprises a sensor data unit in communication with the measurement device, wherein the sensor data unit is configured to receive signals from the sensor representative of the relative axial movement of the second member distal end relative to the first member distal end, and wherein the sensor data is configured to continuously calculate, based on the signals, the axial distance between the inflow end of the prosthetic valve and the first member distal end.
Example 21. The delivery assembly of any example herein, particularly example 20, further comprising a display, wherein the sensor data unit is configured to output an indication of the calculated distance on the display.
Example 22. A delivery assembly, comprising:
a prosthetic valve having an inflow end and an outflow end, the prosthetic valve comprising:
   a frame; and
   a plurality of actuators coupled to support posts of the frame and operable to adjust the frame between a radially compressed and a radially expanded configuration; and
a delivery apparatus comprising:
   a handle;
   a plurality of actuation assemblies extending distally from the handle and configured to operate the plurality of actuators, wherein each actuation assembly comprises an outer sleeve and an actuator driver releasably coupled to a corresponding actuator, the actuator driver extending through the outer sleeve;
   a measurement device comprising:
      a sensor residing within the handle;
      a first motion transmitting member coupled at a first member proximal end thereof to the sensor, and extending from the handle to an opposite first member distal end; and
      one of the plurality of actuator drivers, coupled at a driver proximal end thereof to the sensor;
wherein the first member distal end is a free end; and
wherein the sensor is configured to sense, in real time, axial movement of the actuator
driver attached thereto relative to the first member distal end.
Example 23. The delivery assembly of any example herein, particularly example 22, wherein the sensor is a linear displacement sensor.
Example 24. The delivery assembly of any example herein, particularly example 23, wherein the linear displacement sensor comprises a linear variable differential transformer (LVDT) sensor.
Example 25. The delivery assembly of any example herein, particularly example 24, wherein the LVDT sensor comprises a tube and a transducer core axially movable within the tube, wherein the first member proximal end is attached to the tube, and wherein the driver proximal end is attached to the transducer core.
Example 26. The delivery assembly of any example herein, particularly any one of examples 22 to 25, wherein the delivery apparatus further comprises a shaft assembly comprising an outer delivery shaft, wherein the first motion transmitting member and the actuation assemblies extend through the outer delivery shaft.
Example 27. The delivery assembly of any example herein, particularly example 26, wherein the shaft assembly further comprises a multi-lumen delivery shaft disposed within the outer delivery shaft, and the actuation assemblies extend through separate lumens of the multi-lumen shaft.
Example 28. The delivery assembly of any example herein, particularly example 27, wherein the first motion transmitting member extends through the multi-lumen shaft.
Example 29. The delivery assembly of any example herein, particularly any one of examples 22 to 28, wherein the plurality of actuators comprises six actuators and wherein the plurality of actuation assemblies comprises six actuation assemblies.
Example 30. The delivery assembly of any example herein, particularly any one of examples 22 to 29, wherein each actuator comprises an actuator rod and an attached actuator head.
Example 31. The delivery assembly of any example herein, particularly example 30, wherein the actuator head is disposed at the outflow end of the prosthetic valve.
Example 32. The delivery assembly of any example herein, particularly example 30 or 31, wherein each actuator driver comprises a central protrusion extending into a slot of the corresponding actuator head, and wherein the sensor is configured to sense, in real time, axial movement of the central protrusion of the actuator driver attached thereto relative to the first member distal end.
Example 33. The delivery assembly of any example herein, particularly any one of examples 30 to 32, wherein each actuator rod extends through an upper post member and a lower post member of the frame.
Example 34. The delivery assembly of any example herein, particularly any one of examples 22 to 33, wherein the delivery apparatus further comprises a sensor data unit in communication with the measurement device, wherein the sensor data unit is configured to receive signals from the sensor representative of the relative axial movement of the actuator driver attached thereto relative to the first member distal end, and wherein the sensor data is configured to continuously calculate, based on the signals, the axial distance between the inflow end of the prosthetic valve and the first member distal end.
Example 35. The delivery assembly of any example herein, particularly example 34, wherein the sensor data unit is further configured to receive signals responsive to the number of rotations of at least one of the actuator drivers, and to calculate, based on the counted rotations, the distance between the inflow end and the outflow end.
Example 36. The delivery assembly of any example herein, particularly example 34 or 35, further comprising a display, wherein the sensor data unit is configured to output an indication of the calculated distance on the display.
Example 37. A method, comprising:
positioning a prosthetic valve within a native aortic annulus, the prosthetic valve having an inflow end and an outflow, and comprising a frame movable between a radially compressed and a radially expanded configuration;
advancing a first motion transmitting member of a delivery apparatus toward an aortic sinus, until a first member distal end of the first motion transmitting member contacts a sinus floor of one of the aortic sinuses, the delivery apparatus comprising:
   a handle and a measurement device which comprises: a sensor residing in the handle and attached to a first member proximal end of the first motion transmitting member, and a second motion transmitting member coupled at a second member proximal end thereof to the sensor, and extending from the handle to an opposite second member distal end;
   wherein the second member distal end is releasably coupled to the prosthetic valve;
sensing, by the sensor, in real time, axial movement of the second member distal end relative to the first member distal end.
Example 38. The method of any example herein, particularly example 37, wherein positioning the prosthetic valve within a native aortic annulus comprises positioning the inflow end within the left ventricle, and the outflow end within the aorta.
Example 39. The method of any example herein, particularly example 37 or 38, positioning the prosthetic valve within a native aortic annulus comprises positioning the prosthetic valve in a position such that the first member distal end is disposed at an axial level between the inflow end and the outflow end.
Example 40. The method of any example herein, particularly any one of examples 37 to 39, further comprising sending signals representative of the relative axial movement of the second member distal end relative to the first member distal end, from the sensor to a sensor data unit in communication therewith.
Example 41. The method of any example herein, particularly example 40, wherein further comprising calculating, by the sensor data unit, based on the signals received from the sensor, axial distance between the inflow end of the prosthetic valve and the first member distal end.
Example 42. The method of any example herein, particularly example 41, further comprising outputting an indication of the distance calculated by the sensor data unit, on a display.
Example 43. The method of any example herein, particularly example 42, wherein the display is comprised in the handle.
Example 44. The method of any example herein, particularly any one of examples 41 to 43, further comprising comparing the calculated distance to at least one pre-stored threshold value.
Example 45. The method of any example herein, particularly example 48, wherein the at least one pre-stored threshold value comprises a minimal threshold value and a maximal threshold value.
Example 46. The method of any example herein, particularly any one of examples 41 to 45, wherein sensing by the sensor and calculating the distance by the sensor data unit are performed continuously after the first motion transmitting member contacts the sinus floor.
Example 47. The method of any example herein, particularly example 46, further comprising moving the prosthetic valve between the radially compressed to the radially expanded configuration, wherein sensing by the sensor and calculating the distance by the sensor data unit are performed during and after moving the prosthetic valve between the compressed and expanded configurations.
Example 48. The method of any example herein, particularly example 47, further comprising axially adjusting the position of the prosthetic valve, by moving it in a distal and/or proximal direction within the native aortic annulus, in response to the calculated distance, such that the calculated distance is within a desired pre-defined range.
Example 49. The method of any example herein, particularly any one of examples 37 to 48, wherein the sensor is a linear displacement sensor.
Example 50. The method of any example herein, particularly example 49, wherein the linear displacement sensor comprises a linear variable differential transformer (LVDT) sensor.
Example 51. The method of any example herein, particularly example 50, wherein the LVDT sensor comprises a tube and a transducer core axially movable within the tube, wherein the first member proximal end is attached to the tube, and wherein the second member proximal end is attached to the transducer core.
Example 52. The method of any example herein, particularly any one of examples 37 to 51, wherein the delivery apparatus further comprises a shaft assembly comprising an outer delivery shaft, wherein the first motion transmitting member and the second motion transmitting member extend through the outer delivery shaft.
Example 53. The method of any example herein, particularly example 52, wherein the shaft assembly further comprises a multi-lumen delivery shaft disposed within the outer delivery shaft, and wherein at least one of the first motion transmitting member or the second motion transmitting member extends through the multi-lumen delivery shaft.
Example 54. The method of any example herein, particularly example 53, wherein both the first motion transmitting member and the second motion transmitting member extend through corresponding separate lumens of the multi-lumen delivery shaft.
Example 55. The method of any example herein, particularly example 53, wherein the first motion transmitting member extends through the multi-lumen delivery shaft, and the second motion transmitting member extends through the outer delivery shaft, between an outer surface of the multi-lumen delivery shaft and an inner surface of the outer delivery shaft.
Example 56. The method of any example herein, particularly any one of examples 37 to 55, wherein the second member distal end is releasably coupled to the inflow end of the prosthetic valve.
Example 57. The method of any example herein, particularly example 56, wherein the second member distal end is releasably coupled to an inflow apex of the prosthetic valve.
Example 58. The method of any example herein, particularly any one of examples 37 to 57, wherein the second member distal end is releasably coupled to the frame.
Example 59. The method of any example herein, particularly example 58, wherein the frame comprises a plurality of support posts aligned with a longitudinal axis of the prosthetic valve, and wherein the second member distal end is releasably coupled to one of the support posts.
Example 60. The method of any example herein, particularly example 59, wherein the support post to which the second member distal end is attached, further comprises a commissure window.
Example 61. The method of any example herein, particularly example 59, wherein the support post to which the second member distal end is attached, further comprises a cantilevered strut extending toward the inflow end.
Example 62. The method of any example herein, particularly example 59, wherein at least one of the support posts comprises an upper post member and a lower post member, and wherein the second member distal end is releasably coupled to the lower post member.
Example 63. The method of any example herein, particularly any one of examples 37 to 62, wherein the second member distal end is glued to the prosthetic valve.
Example 64. The method of any example herein, particularly any one of examples 37 to 62, wherein the prosthetic valve further comprises a coupler attached to the frame, and wherein the second member distal end is threadedly engaged with the coupler.
Example 65. The method of any example herein, particularly example 64, wherein the coupler comprises a threaded bore, and wherein the second member distal end comprises an outer threading.
Example 66. The method of any example herein, particularly any one of examples 37 to 65, wherein further comprising decoupling the second member distal end from the prosthetic valve, and retrieving the delivery assembly by pulling it proximally away from the prosthetic valve.
Example 67. The method of any example herein, particularly example 66, wherein, when depending on example 63, decoupling the second member distal end comprises forcibly pulling the second motion transmitting member relative to the prosthetic valve
Example 68. The method of any example herein, particularly example 66, wherein, when depending on example 64, decoupling the second member distal end comprises rotating the second motion transmitting member in a direction that threadedly disengages it from the coupler.
Example 69. The method of any example herein, particularly any one of examples 37 to 68, wherein the prosthetic valve further comprises a plurality of actuators coupled to support posts of the frame and operable to move the frame between the radially compressed and the radially expanded configuration, and wherein the delivery assembly further comprises a plurality of actuation assemblies extending distally from the handle and configured to operate the plurality of actuators, wherein each actuation assembly comprises an outer sleeve and an actuator driver releasably coupled to a corresponding actuator, the actuator driver extending through the outer sleeve.
Example 70. The method of any example herein, particularly example 69, wherein, when depending on claim 47, moving the prosthetic valve between the compressed and expanded configurations comprises actuating, via one or more knobs of the handle, the plurality of actuation assemblies.
Example 71. The method of any example herein, particularly example 70, wherein actuating the actuation assemblies comprises rotating the plurality of actuator drivers.
Example 72. The method of any example herein, particularly example 69, wherein, when depending on example 66, further comprises decoupling the actuation assemblies from the corresponding actuators prior to retrieving the delivery apparatus.
Example 73. The method of any example herein, particularly any one of examples 37 to 68, wherein the delivery assembly further comprises a balloon catheter with an inflatable balloon.
Example 74. The method of any example herein, particularly example 73, wherein positioning a prosthetic valve within a native aortic annulus further comprises positioning the prosthetic valve, in its compressed configuration, over the balloon, in a deflated state of the balloon.
Example 75. The method of any example herein, The method of any example herein, particularly example 73 or 74, wherein, when depending on example 47, moving the prosthetic valve between the compressed and expanded configurations comprises inflating the balloon.
Example 76. A delivery assembly, comprising:
a prosthetic valve having an inflow end and an outflow end, the prosthetic valve comprising:
   a frame; and
   a plurality of actuators coupled to support posts of the frame and operable to adjust the frame between a radially compressed and a radially expanded configuration; and
a delivery apparatus comprising:
   a handle;
   a plurality of actuation assemblies extending distally from the handle and configured to operate the plurality of actuators, wherein each actuation assembly comprises an outer sleeve and an actuator driver releasably coupled to a corresponding actuator, the actuator driver extending through the outer sleeve;
   a measurement device comprising:
      a sensor residing within the handle;
      a first motion transmitting member coupled at a first member proximal end thereof to the sensor, and extending from the handle to an opposite first member distal end; and
      a second motion transmitting member coupled at a second member proximal end thereof to the sensor, and extending from the handle to an opposite second member distal end;
wherein the first member distal end is a free end;
wherein the second member distal end is attached to one of the plurality of actuation assemblies; and
wherein the sensor is configured to sense, in real time, axial movement of the second member distal end relative to the first member distal end.
Example 77. The method of any example herein, particularly example 76, wherein the sensor is a linear displacement sensor.
Example 78. The method of any example herein, particularly example 77, wherein the linear displacement sensor comprises a linear variable differential transformer (LVDT) sensor.
Example 79. The method of any example herein, particularly example 78, wherein the LVDT sensor comprises a tube and a transducer core axially movable within the tube, wherein the first member proximal end is attached to the tube, and wherein the second member proximal end is attached to the transducer core.
Example 80. The delivery assembly of any example herein, particularly any one of examples 76 to 79, wherein the delivery apparatus further comprises a shaft assembly comprising an outer delivery shaft, wherein the first motion transmitting member and the actuation assemblies extend through the outer delivery shaft.
Example 81. The method of any example herein, particularly example 80, wherein the shaft assembly further comprises a multi-lumen delivery shaft disposed within the outer delivery shaft, and the actuation assemblies extend through separate lumens of the multi-lumen shaft, and wherein the second motion transmitting member extends through the same lumen of the actuation assembly it is attached to.
Example 82. The method of any example herein, particularly example 81, wherein the first motion transmitting member extends through the multi-lumen shaft.
Example 83. The delivery assembly of any example herein, particularly any one of examples 76 to 82, wherein the plurality of actuators comprises six actuators and wherein the plurality of actuation assemblies comprises six actuation assemblies.
Example 84. The delivery assembly of any example herein, particularly any one of examples 76 to 83, wherein the second member distal end is coupled to the actuator driver of the actuation assembly.
Example 85. The method of any example herein, particularly example 84, wherein the second member distal end is coupled to a central protrusion of the actuator driver.
Example 86. The delivery assembly of any example herein, particularly any one of examples 76 to 83, wherein the second member distal end is coupled to the outer sleeve of the actuation assembly.
Example 87. The delivery assembly of any example herein, particularly any one of examples 76 to 86, wherein each actuator comprises an actuator rod and an attached actuator head.
Example 88. The method of any example herein, particularly example 87, wherein the actuator head is disposed at the outflow end of the prosthetic valve.
Example 89. The delivery assembly of any example herein, particularly example 87 or 88, wherein each actuator rod extends through an upper post member and a lower post member of the frame.
Example 90. The delivery assembly of any example herein, particularly any one of examples 76 to 89, wherein the delivery apparatus further comprises a sensor data unit in communication with the measurement device, wherein the sensor data unit is configured to receive signals from the sensor representative of the relative axial movement of the second member distal end relative to the first member distal end, and wherein the sensor data is configured to continuously calculate, based on the signals, the axial distance between the inflow end of the prosthetic valve and the first member distal end.
Example 91. The method of any example herein, particularly example 90, wherein the sensor data unit is further configured to receive signals responsive to the number of rotations of at least one of the actuator drivers, and to calculate, based on the counted rotations, the distance between the inflow end and the outflow end.
Example 92. The method of any example herein, particularly example 90 or 91, further comprising a display, wherein the sensor data unit is configured to output an indication of the calculated distance on the display.
Example 93. A method, comprising:
positioning a prosthetic valve within a native aortic annulus, the prosthetic valve having an inflow end and an outflow, and comprising:
   a frame; and
   a plurality of actuators coupled to support posts of the frame and operable to adjust the frame between a radially compressed and a radially expanded configuration;
advancing a first motion transmitting member of a delivery apparatus toward an aortic sinus, until a first member distal end of the first motion transmitting member contacts a sinus floor of one of the aortic sinuses, the delivery apparatus comprising:
   a handle;
   a plurality of actuation assemblies extending distally from the handle and configured to operate the plurality of actuators, wherein each actuation assembly comprises an outer sleeve and an actuator driver releasably coupled to a corresponding actuator, the actuator driver extending through the outer sleeve;
   a measurement device comprising:
      a sensor residing within the handle;
      a first motion transmitting member coupled at a first member proximal end thereof to the sensor, and extending from the handle to an opposite first member distal end;
      one of the plurality of actuator drivers, coupled at a driver proximal end thereof to the sensor;
sensing, by the sensor, in real time, axial movement of the actuator driver attached thereto relative to the first member distal end.
Example 94. The method of any example herein, particularly example 93, wherein positioning the prosthetic valve within a native aortic annulus comprises positioning the inflow end within the left ventricle, and the outflow end within the aorta.
Example 95. The method of any example herein, particularly example 93 or 94, wherein positioning the prosthetic valve within a native aortic annulus comprises positioning the prosthetic valve in a position such that the first member distal end is disposed at an axial level between the inflow end and the outflow end.
Example 96. The method of any example herein, particularly any one of examples 93 to 95, wherein further comprising sending signals representative of the relative axial movement of the actuator driver relative to the first member distal end, from the sensor to a sensor data unit in communication therewith.
Example 97. The method of any example herein, particularly example 96, further comprising calculating, by the sensor data unit, based on the signals received from the sensor, axial distance between the inflow end of the prosthetic valve and the first member distal end.
Example 98. The method of any example herein, particularly example 97, wherein the step of calculating axial distance between the inflow end of the prosthetic valve and the first member distal end comprises:
calculating, by the sensor data unit, based on the signals received from the sensor, axial distance between the outflow end of the prosthetic valve and the first member distal end; and
calculating, by the sensor data unit, based on the signals responsive to the number of rotations of at least one actuator driver, and pre-stored relationships between number of rotations and relative axial movement between the inflow end and the outflow end, axial distance between the inflow end and the outflow end of the prosthetic valve.
Example 99. The method of any example herein, particularly example 97, further comprising outputting an indication of the distance calculated by the sensor data unit, on a display.
Example 100. The method of any example herein, particularly example 97, wherein the display is comprised in the handle.
Example 101. The method of any example herein, particularly any one of examples 97 to 100, further comprising comparing the calculated distance to at least one pre-stored threshold value.
Example 102. The method of any example herein, particularly example 101, wherein the at least one pre-stored threshold value comprises a minimal threshold value and a maximal threshold value.
Example 103. The method of any example herein, particularly any one of examples 97 to 102, wherein sensing by the sensor and calculating the distance by the sensor data unit are performed continuously after the first motion transmitting member contacts the sinus floor.
Example 104. The method of any example herein, particularly example 103, further comprising moving the prosthetic valve between the radially compressed to the radially expanded configuration, wherein sensing by the sensor and calculating the distance by the sensor data unit are performed during and after moving the prosthetic valve between the compressed and expanded configurations.
Example 105. The method of any example herein, particularly example 104, further comprising axially adjusting the position of the prosthetic valve, by moving it in a distal and/or proximal direction within the native aortic annulus, in response to the calculated distance, such that the calculated distance is within a desired pre-defined range.
Example 106. The method of any example herein, particularly any one of examples 93 to 105, wherein the sensor is a linear displacement sensor.
Example 107. The method of any example herein, particularly example 106, wherein the linear displacement sensor comprises a linear variable differential transformer (LVDT) sensor.
Example 108. The method of any example herein, particularly example 107, wherein the LVDT sensor comprises a tube and a transducer core axially movable within the tube, wherein the first member proximal end is attached to the tube, and wherein the driver proximal end is attached to the transducer core.
Example 109. The method of any example herein, particularly any one of examples 93 to 108, wherein the delivery apparatus further comprises a shaft assembly comprising an outer delivery shaft, wherein the first motion transmitting member and the actuation assemblies extend through the outer delivery shaft.
Example 110. The method of any example herein, particularly example 109, wherein the shaft assembly further comprises a multi-lumen delivery shaft disposed within the outer delivery shaft, and the actuation assemblies extend through separate lumens of the multi-lumen shaft.
Example 111. The method of any example herein, particularly example 110, wherein the first motion transmitting member extends through the multi-lumen shaft.
Example 112. The method of any example herein, particularly any one of examples 93 to 111, wherein the plurality of actuators comprises six actuators and wherein the plurality of actuation assemblies comprises six actuation assemblies.
Example 113. The method of any example herein, particularly any one of examples 93 to 112, wherein each actuator comprises an actuator rod and an attached actuator head.
Example 114. The method of any example herein, particularly example 113, wherein the actuator head is disposed at the outflow end of the prosthetic valve.
Example 115. The method of any example herein, particularly example 113 or 114, wherein each actuator driver comprises a central protrusion extending into a slot of the corresponding actuator head, and wherein the sensor is configured to sense, in real time, axial movement of the central protrusion of the actuator driver attached thereto relative to the first member distal end.
Example 116. The method of any example herein, particularly any one of examples 113 to 115, wherein each actuator rod extends through an upper post member and a lower post member of the frame.
Example 117. The method of any example herein, particularly example 104, wherein moving the prosthetic valve between the compressed and expanded configurations comprises actuating, by at least one knob of the handle, the plurality of actuation assemblies.
Example 118. The method of any example herein, particularly example 117, wherein actuating the actuation assemblies comprises rotating the actuator drivers.
Example 119. The method of any example herein, particularly any one of examples 93 to 118, further comprising decoupling the actuation assemblies from the prosthetic valve, and retrieving the delivery assembly by pulling it proximally away from the prosthetic valve.
Example 120. A method, comprising:
positioning a prosthetic valve within a native aortic annulus, the prosthetic valve having an inflow end and an outflow, and comprising:
   a frame; and
   a plurality of actuators coupled to support posts of the frame and operable to adjust the frame between a radially compressed and a radially expanded configuration;
advancing a first motion transmitting member of a delivery apparatus toward an aortic sinus, until a first member distal end of the first motion transmitting member contacts a sinus floor of one of the aortic sinuses, the delivery apparatus comprising:
   a handle; and
   a plurality of actuation assemblies extending distally from the handle and configured to operate the plurality of actuators, wherein each actuation assembly comprises an outer sleeve and an actuator driver releasably coupled to a corresponding actuator, the actuator driver extending through the outer sleeve;
   a measurement device comprising:
      a sensor residing within the handle;
      a first motion transmitting member coupled at a first member proximal end thereof to the sensor, and extending from the handle to an opposite first member distal end; and
      a second motion transmitting member coupled at a second member proximal end thereof to the sensor, and extending from the handle to an opposite second member distal end;
   wherein the second member distal end is attached to one of the plurality of actuation assemblies;
sensing, by the sensor, in real time, axial movement of the second member distal end relative to the first member distal end.
Example 121. The method of any example herein, particularly example 120, wherein positioning the prosthetic valve within a native aortic annulus comprises positioning the inflow end within the left ventricle, and the outflow end within the aorta.
Example 122. The method of any example herein, particularly example 120 or 121, wherein positioning the prosthetic valve within a native aortic annulus comprises positioning the prosthetic valve in a position such that the first member distal end is disposed at an axial level between the inflow end and the outflow end.
Example 123. The method of any example herein, particularly any one of examples 120 to 122, further comprising sending signals representative of the relative axial movement of the second member distal end relative to the first member distal end, from the sensor to a sensor data unit in communication therewith.
Example 124. The method of any example herein, particularly example 123, further comprising calculating, by the sensor data unit, based on the signals received from the sensor, axial distance between the inflow end of the prosthetic valve and the first member distal end.
Example 125. The method of any example herein, particularly example 124, wherein the step of calculating axial distance between the inflow end of the prosthetic valve and the first member distal end comprises:
calculating, by the sensor data unit, based on the signals received from the sensor, axial distance between the outflow end of the prosthetic valve and the first member distal end; and
calculating, by the sensor data unit, based on signals responsive to the number of rotations of at least one actuator driver, and pre-stored relationships between number of rotations and relative axial movement between the inflow end and the outflow end, axial distance between the inflow end and the outflow end of the prosthetic valve.
Example 126. The method of any example herein, particularly example 124 or 125, further comprising outputting an indication of the distance calculated by the sensor data unit, on a display.
Example 127. The method of any example herein, particularly example 126, wherein the display is comprised in the handle.
Example 128. The method of any example herein, particularly any one of examples 124 to 127, further comprising comparing the calculated distance to at least one pre-stored threshold value.
Example 129. The method of any example herein, particularly example 128, wherein the at least one pre-stored threshold value comprises a minimal threshold value and a maximal threshold value.
Example 130. The method of any example herein, particularly any one of examples 124 to 129, wherein sensing by the sensor and calculating the distance by the sensor data unit are performed continuously after the first motion transmitting member contacts the sinus floor.
Example 131. The method of any example herein, particularly example 130, further comprising moving the prosthetic valve between the radially compressed to the radially expanded configuration, wherein sensing by the sensor and calculating the distance by the sensor data unit are performed during and after moving the prosthetic valve between the compressed and expanded configurations.
Example 132. The method of any example herein, particularly example 131, further comprising axially adjusting the position of the prosthetic valve, by moving it in a distal and/or proximal direction within the native aortic annulus, in response to the calculated distance, such that the calculated distance is within a desired pre-defined range.
Example 133. The method of any example herein, particularly any one of examples 120 to 132, wherein the sensor is a linear displacement sensor.
Example 134. The method of any example herein, particularly example 133, wherein the linear displacement sensor comprises a linear variable differential transformer (LVDT) sensor.
Example 135. The method of any example herein, particularly example 134, wherein the LVDT sensor comprises a tube and a transducer core axially movable within the tube, wherein the first member proximal end is attached to the tube, and wherein the second member proximal end is attached to the transducer core.
Example 136. The method of any example herein, particularly any one of examples 120 to 135, wherein the delivery apparatus further comprises a shaft assembly comprising an outer delivery shaft, wherein the first motion transmitting member and the actuation assemblies extend through the outer delivery shaft.
Example 137. The method of any example herein, particularly example 136, wherein the shaft assembly further comprises a multi-lumen delivery shaft disposed within the outer delivery shaft, and the actuation assemblies extend through separate lumens of the multi-lumen shaft, and wherein the second motion transmitting member extends through the same lumen of the actuation assembly it is attached to.
Example 138. The method of any example herein, particularly example 137, wherein the first motion transmitting member extends through the multi-lumen shaft.
Example 139. The method of any example herein, particularly any one of examples 120 to 138, wherein the plurality of actuators comprises six actuators and wherein the plurality of actuation assemblies comprises six actuation assemblies.
Example 140. The method of any example herein, particularly any one of examples 120 to 139, wherein the second member distal end is coupled to the actuator driver of the actuation assembly.
Example 141. The method of any example herein, particularly example 140, wherein the second member distal end is coupled to a central protrusion of the actuator driver.
Example 142. The method of any example herein, particularly any one of examples 120 to 139, wherein the second member distal end is coupled to the outer sleeve of the actuation assembly.
Example 143. The method of any example herein, particularly any one of examples 120 to 142, wherein each actuator comprises an actuator rod and an attached actuator head.
Example 144. The method of any example herein, particularly example 143, wherein the actuator head is disposed at the outflow end of the prosthetic valve.
Example 145. The method of any example herein, particularly example 143 or 144, wherein each actuator rod extends through an upper post member and a lower post member of the frame.
Example 146. The method of any example herein, particularly example 131, wherein moving the prosthetic valve between the compressed and expanded configurations comprises actuating, by at least one knob of the handle, the plurality of actuation assemblies.
Example 147. The method of any example herein, particularly example 146, wherein actuating the actuation assemblies comprises rotating the actuator drivers.
Example 148. The method of any example herein, particularly any one of examples 120 to 147, wherein further comprising decoupling the actuation assemblies from the prosthetic valve, and retrieving the delivery assembly by pulling it proximally away from the prosthetic valve.
Example 149. A delivery assembly, comprising:
a prosthetic valve having an inflow end and an outflow end, the prosthetic valve comprising a frame movable between a radially compressed and a radially expanded configuration; and
a delivery apparatus comprising:
   a handle;
   a balloon catheter extending from the handle and comprising an inflatable balloon mounted on a distal end thereof; and
   a push shaft extending from the handle and disposed over the balloon catheter, the push shaft comprising a push shaft distal end portion opposite to the handle;
   a measurement device comprising:
      a sensor residing within the handle;
      a first motion transmitting member coupled at a first member proximal end thereof to the sensor, and extending from the handle to an opposite first member distal end; and
      a second motion transmitting member coupled at a second member proximal end thereof to the sensor, and extending from the handle to an opposite second member distal end;
wherein the first member distal end is a free end;
wherein the second member distal end is coupled to the push shaft; and
wherein the sensor is configured to sense, in real time, axial movement of the second member distal end relative to the first member distal end.
Example 150. The method of any example herein, particularly example 149, wherein the sensor is a linear displacement sensor.
Example 151. The method of any example herein, particularly example 150, wherein the linear displacement sensor comprises a linear variable differential transformer (LVDT) sensor.
Example 152. The method of any example herein, particularly example 151, wherein the LVDT sensor comprises a tube and a transducer core axially movable within the tube, wherein the first member proximal end is attached to the tube, and wherein the second member proximal end is attached to the transducer core.
Example 153. The delivery assembly of any example herein, particularly any one of examples 149 to 152, wherein the second member distal end is coupled to the push shaft distal end portion.
Example 154. The delivery assembly of any example herein, particularly any one of examples 149 to 153, further comprising an outer delivery shaft disposed over the push shaft.
Example 155. The method of any example herein, particularly example 151, wherein the first motion transmitting member extends through the outer delivery shaft.
Example 156. The delivery assembly of any example herein, particularly any one of examples 149 to 155, wherein the first motion transmitting member extends through the outer delivery shaft.
Example 157. The delivery assembly of any example herein, particularly any one of examples 149 to 156, wherein the push shaft distal end portion is dimensioned to engage with the outflow end of the prosthetic valve in a crimped configuration of the prosthetic valve.
Example 158. The method of any example herein, particularly example 157, wherein the push shaft distal end portion is flared radially outward.
Example 159. The delivery assembly of any example herein, particularly any one of examples 149 to 158, wherein the delivery apparatus further comprises a sensor data unit in communication with the measurement device, wherein the sensor data unit is configured to receive signals from the sensor representative of the relative axial movement of the second member distal end relative to the first member distal end, and wherein the sensor data is configured to continuously calculate, based on the signals, the axial distance between the inflow end of the prosthetic valve and the first member distal end.
Example 160. The method of any example herein, particularly example 159, further comprising a display, wherein the sensor data unit is configured to output an indication of the calculated distance on the display.
Example 161. A method, comprising:
delivering a prosthetic valve in a radially compressed configuration thereof toward a native aortic annulus, while the prosthetic valve is positioned over a balloon catheter of a delivery apparatus, proximal to an inflatable balloon mounted on a distal end of the balloon catheter, wherein the prosthetic valve has an inflow end and an outflow, and comprises a frame movable between the radially compressed and a radially expanded configuration, and wherein the delivery apparatus further comprises:
the delivery apparatus comprising:
   a handle;
   a balloon catheter extending from the handle and comprising an inflatable balloon mounted on a distal end thereof;
   a push shaft extending from the handle and disposed over the balloon catheter, the push shaft comprising a push shaft distal end portion opposite to the handle;
   a measurement device comprising:
      a sensor residing within the handle;
      a first motion transmitting member coupled at a first member proximal end thereof to the sensor, and extending from the handle to an opposite first member distal end;
      a second motion transmitting member coupled at a second member proximal end thereof to the sensor, and extending from the handle to an opposite second member distal end;
   wherein the second member distal end is coupled to the prosthetic valve;
advancing the first motion transmitting member toward an aortic sinus, until the first member distal end contacts a sinus floor of one of the aortic sinuses;
pushing, by the push shaft, the prosthetic valve to position it over the balloon;
positioning the prosthetic valve within the native aortic annulus; and
sensing, by the sensor, in real time, axial movement of the second member distal end relative to the first member distal end.
Example 162. The method of any example herein, particularly example 161, wherein positioning the prosthetic valve within a native aortic annulus comprises positioning the inflow end within the left ventricle, and the outflow end within the aorta.
Example 163. The method of any example herein, particularly example 161 or 162, wherein positioning the prosthetic valve within a native aortic annulus comprises positioning the prosthetic valve in a position such that the first member distal end is disposed at an axial level between the inflow end and the outflow end.
Example 164. The method of any example herein, particularly any one of examples 161 to 163, further comprising sending signals representative of the relative axial movement of the second member distal end relative to the first member distal end, from the sensor to a sensor data unit in communication therewith.
Example 165. The method of any example herein, particularly example 164, further comprising calculating, by the sensor data unit, based on the signals received from the sensor, axial distance between the inflow end of the prosthetic valve and the first member distal end.
Example 166. The method of any example herein, particularly example 165, wherein the step of calculating axial distance between the inflow end of the prosthetic valve and the first member distal end comprises:
calculating, by the sensor data unit, based on the signals received from the sensor, axial distance between the outflow end of the prosthetic valve and the first member distal end; and
calculating, by the sensor data unit, based on signals responsive to amount of balloon inflation and/or prosthetic valve expansion, and pre-stored relationships between balloon inflation and/or prosthetic valve expansion and prosthetic valve foreshortening, axial distance between the inflow end and the outflow end of the prosthetic valve.
Example 167. The method of any example herein, particularly example 165 or 166, further comprising outputting an indication of the distance calculated by the sensor data unit, on a display.
Example 168. The method of any example herein, particularly example 167, wherein the display is comprised in the handle.
Example 169. The method of any example herein, particularly any one of examples 165 to 168, further comprising comparing the calculated distance to at least one pre-stored threshold value.
Example 170. The method of any example herein, particularly example 169, wherein the at least one pre-stored threshold value comprises a minimal threshold value and a maximal threshold value.
Example 171. The method of any example herein, particularly any one of examples 165 to 167, wherein sensing by the sensor and calculating the distance by the sensor data unit are performed continuously after the first motion transmitting member contacts the sinus floor.
Example 172. The method of any example herein, particularly example 171, further comprising inflating the balloon to transition the prosthetic valve to the expanded configuration.
Example 173. The method of any example herein, particularly example 171 or 172, further comprising axially adjusting the position of the prosthetic valve, by moving it in a distal and/or proximal direction within the native aortic annulus, in response to the calculated distance, such that the calculated distance is within a desired pre-defined range.
Example 174. The method of any example herein, particularly any one of examples 161 to 173, wherein the sensor is a linear displacement sensor.
Example 175. The method of any example herein, particularly example 174, wherein the linear displacement sensor comprises a linear variable differential transformer (LVDT) sensor.
Example 176. The method of any example herein, particularly example 175, wherein the LVDT sensor comprises a tube and a transducer core axially movable within the tube, wherein the first member proximal end is attached to the tube, and wherein the second member proximal end is attached to the transducer core.
Example 177. The method of any example herein, particularly example 176, further comprising deflating the balloon and retrieving the delivery apparatus by pulling it proximally away from the prosthetic valve.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A delivery assembly, comprising:
a prosthetic valve having an inflow end and an outflow end, the prosthetic valve comprising a frame movable between a radially compressed and a radially expanded configuration; and
a delivery apparatus comprising:
a handle;
a measurement device comprising:
a sensor residing within the handle;
a first motion transmitting member coupled at a first member proximal end thereof to the sensor, and extending from the handle to an opposite first member distal end; and
a second motion transmitting member coupled at a second member proximal end thereof to the sensor, and extending from the handle to an opposite second member distal end;
wherein the first member distal end is a free end;
wherein the second member distal end is releasably coupled to the prosthetic valve; and
wherein the sensor is configured to sense, in real time, axial movement of the second member distal end relative to the first member distal end.

2. The delivery assembly of claim 1, wherein the sensor is a linear displacement sensor.

3. The delivery assembly of claim 1, wherein the second member distal end is releasably coupled to the inflow end of the prosthetic valve.

4. The delivery assembly of claim 1, wherein the second member distal end is releasably coupled to the frame.

5. The delivery assembly of claim 1, wherein the prosthetic valve further comprises a coupler attached to the frame, and wherein the second member distal end is threadedly engaged with the coupler.

6. The delivery assembly of claim 1, wherein the delivery apparatus further comprises a sensor data unit in communication with the measurement device, wherein the sensor data unit is configured to receive signals from the sensor representative of the relative axial movement of the second member distal end relative to the first member distal end, and wherein the sensor data is configured to continuously calculate, based on the signals, the axial distance between the inflow end of the prosthetic valve and the first member distal end.

7. The delivery assembly of claim 6, further comprising a display, wherein the sensor data unit is configured to output an indication of the calculated distance on the display.

8. A method comprising:
positioning a prosthetic valve within a native aortic annulus, the prosthetic valve having an inflow end and an outflow, and comprising a frame movable between a radially compressed and a radially expanded configuration;
advancing a first motion transmitting member of a delivery apparatus toward an aortic sinus, until a first member distal end of the first motion transmitting member contacts a sinus floor of one of the aortic sinuses, the delivery apparatus comprising:
a handle and a measurement device which comprises: a sensor residing in the handle and attached to a first member proximal end of the first motion transmitting member, and a second motion transmitting member coupled at a second member proximal end thereof to the sensor, and extending from the handle to an opposite second member distal end;
wherein the second member distal end is releasably coupled to the prosthetic valve; and
sensing, by the sensor, in real time, axial movement of the second member distal end relative to the first member distal end.

9. The method of claim 8, wherein positioning the prosthetic valve within a native aortic annulus comprises positioning the prosthetic valve in a position such that the first member distal end is disposed at an axial level between the inflow end and the outflow end.

10. The method of claim 8, further comprising sending signals representative of the relative axial movement of the second member distal end relative to the first member distal end, from the sensor to a sensor data unit in communication therewith.

11. The method of claim 10, further comprising calculating, by the sensor data unit, based on the signals received from the sensor, axial distance between the inflow end of the prosthetic valve and the first member distal end.

12. The method of claim 11, further comprising comparing the calculated distance to at least one pre-stored threshold value.

13. The method of claim 11, wherein sensing by the sensor and calculating the distance by the sensor data unit are performed continuously after the first motion transmitting member contacts the sinus floor.

14. The method of claim 13, further comprising moving the prosthetic valve between the radially compressed to the radially expanded configuration, wherein sensing by the sensor and calculating the distance by the sensor data unit are performed during and after moving the prosthetic valve between the compressed and expanded configurations.

15. The method of claim 14, further comprising axially adjusting the position of the prosthetic valve, by moving it in a distal and/or proximal direction within the native aortic annulus, in response to the calculated distance, such that the calculated distance is within a desired pre-defined range.

16. The method of claim 8, further comprising decoupling the second member distal end from the prosthetic valve, and retrieving the delivery assembly by pulling it proximally away from the prosthetic valve.

17. A delivery assembly, comprising:
a prosthetic valve having an inflow end and an outflow end, the prosthetic valve comprising a frame movable between a radially compressed and a radially expanded configuration; and
a delivery apparatus comprising:
a handle;
a balloon catheter extending from the handle and comprising an inflatable balloon mounted on a distal end thereof; and
a push shaft extending from the handle and disposed over the balloon catheter, the push shaft comprising a push shaft distal end portion opposite to the handle;
a measurement device comprising:
a sensor residing within the handle;
a first motion transmitting member coupled at a first member proximal end thereof to the sensor, and extending from the handle to an opposite first member distal end; and
a second motion transmitting member coupled at a second member proximal end thereof to the sensor, and extending from the handle to an opposite second member distal end;
wherein the first member distal end is a free end;
wherein the second member distal end is coupled to the push shaft; and
wherein the sensor is configured to sense, in real time, axial movement of the second member distal end relative to the first member distal end.

18. The delivery assembly of claim 17, wherein the push shaft distal end portion is proximal to the outflow end of the prosthetic valve.

19. The delivery assembly of claim 17, wherein the delivery apparatus further comprises a sensor data unit in communication with the measurement device, wherein the sensor data unit is configured to receive signals from the sensor representative of the relative axial movement of the second member distal end relative to the first member distal end, and wherein the sensor data is configured to continuously calculate, based on the signals, the axial distance between the inflow end of the prosthetic valve and the first member distal end.

20. The delivery assembly of claim 19, further comprising a display, wherein the sensor data unit is configured to output an indication of the calculated distance on the display.
